(19) United States
(12) Patent Application Publication
(10) Pub. No.: US 2007/0287881 A1
(43) Pub. Date: Dec. 13, 2007

(54) Destressing system, apparatus, and method therefor

(76) Inventors: Anatoly E. Akimov, Moscow (RU); Anita B. Kersten, Granby, CT (US); Robert R. Kersten, Granby, CT (US); Marc S. Newkirk, Chester, MA (US); Vera G. Akimov, Moscow (RU)

Correspondence Address: PAUL, HASTINGS, JANOFSKY & WALKER LLP, 875 15th Street, NW, Washington, DC, 20005, US

(21) Appl. No.: 11/783,813

(22) Filed: Apr. 12, 2007

Related U.S. Application Data
(60) Provisional application No. 60/791,964, filed on Apr. 13, 2006.

(51) Int. Cl. A61M 21/00

(52) U.S. Cl. 600/26; 600/27; 600/27

(57) ABSTRACT

A device, system, apparatus, and method are disclosed for reducing stress in an individual by creating an enhanced informational spin field environment substantially surrounding the individual. Such informational spin field environment is at least partially derived from one or more dynamically produced informational spin fields wherein electromagnetic components associated with producing one or more of such dynamically produced informational spin fields are blocked from propagating therewith, such one or more dynamically produced informational spin fields being then conducted without accompanying electromagnetic signals to the environment substantially surrounding the individual.

This application claims the benefit of U.S. Provisional Application No. 60/791,964, filed Apr. 13, 2006, which is herein incorporated by reference in its entirety.

### BACKGROUND

1. Field of the Invention

This invention relates to a method, apparatus, and system for reducing stress in an individual.

2. Background of the Invention

The increasing complexity and population density of our society seems to be increasingly conducive to the creation of stress in the population. There has appeared, therefore, a growing need to identify more effective means of alleviating stress, and as a result a variety of new therapies and technologies for dealing with stress have surfaced over the past century.

Stress is viewed as the cause of many forms of unhappiness in people, such as irritability, depression, anger, emotional instability, withdrawal, restlessness, anxiety and frustration, and dysfunction in all living beings. The link between stress and health is well known. The Journal of Occupational and Environmental Medicine observes that health care expenditures are nearly 50% greater for workers who report high levels of stress. Medical symptoms widely attributed to stress include increased heart rate and blood pressure, headache, nausea, indigestion, and insomnia. In fact, the onslaught of disease more generally is increasingly being related to stress. The American Institute of Stress, founded in 1978 by such notables as Linus Pauling, Alvin Toffler, Herbert Benson, and numerous other prominent scientists and physicians, currently describes stress as “America's No. 1 health problem.” In answer to the question as to how stress can cause so many diseases, the Institute states, “many of these effects are due to increased sympathetic nervous system activity.”

It is well known that stress can be relieved in humans by rest, and by resorting to natural environments such as lakes, seashores, mountains, gardens, and forests. It is also well known that spas, soft lights and certain types of sound or music can relieve stress. In some cases light and color have been observed to have benefits with respect to both stress alleviation and healing. Spectro-Chrome, a colored light therapy introduced in 1920 by Dinshah Ghadiali, developed an impressive array of successes in healing a wide range of diseases over a thirty year period. Sound healing CDs have been produced by Andrew Weil, M.D., founder of the Program in Integrative Medicine at the University of Arizona, and Mitchell Gaynor, M.D., founder and president of Gaynor Integrative Oncology in New York City.

A device invented and patented by Barry McNew (U.S. Patent No. 6,544,165) uses a combination of music and light to accomplish stress reduction and healing. An individual lies in a horizontal cabinet designed to resonate with sound corresponding to a B minor (C flat minor) chord. Successful clinical results for this device are described in the Proceedings of the First Interdisciplinary International Conference on the Science of Whole Person Healing. McNew's device is specifically described as being directed at balancing the sympathetic and parasympathetic elements of the autonomic nervous system. McNew's international patent application, published under the Patent Cooperation Treaty (WO 2005/058144 & PCT/US2004/042451), describes the use of indicia such as involuntary eye or foot movements as references for the operator in adjusting sound and light inputs to the device to accomplish balancing the environment within the device to achieve the desired effect. Destressing is specifically claimed as an attribute of the device, with supporting evidence being accumulated on numerous subjects using HRV monitoring before and after exposure to the device. A typical exposure of an individual in the device is described as one hour at a session.

The past two decades have seen an increasing recognition by scientists of the existence of a new fundamental field in physics beyond the long recognized electrical, magnetic, gravitational, and strong and weak nuclear attraction fields, namely, the informational field (IF), with characteristics unique as compared with the classical fields. An example of an informational field is shown in the conservation of twin photons in entanglement experiments, where the transfer of information necessary to conserve spin can happen without energetic properties.

This more newly recognized field has been described by other names as well, such as torsion field, spin field, and informational spin field. The seminal work in understanding and demonstrating the reality of informational fields was done in the former Soviet Union by Russian physicist Anatoly E. Akimov, a coinventor of the present invention, and Russian theoretical physicist Gennady I. Shipov, both of the International Institute of Theoretical and Applied Physics of the Russian Academy of Natural Sciences. A summary of the theory and numerous technologies created as a result of this discovery appears in Dr. Akimov's paper delivered in Moscow in 2000, entitled, Horizons of XXI Century Science and Technologies. A description of the mathematical basis further elaborating and supporting the theory is described in Dr. Shipov's book, A Theory of Physical Vacuum—A New Paradigm, published in Russian in 1993, and in English in 1998.

In the experimental work with informational spin fields (ISFs), ISFs were found to have different properties than known classical fields. For example, they do not decrease with distance, as all of the other known fields do, according to the inverse square law. ISFs have a spatial structure corresponding to axial symmetry. Objects with like (left-oriented or right-oriented) spins attract, unlike objects with like electrical charges, which repel. ISFs are capable of spin-polarizing space, such that even when a source of an ISF is removed, the space where the field was tends to retain its ISF-influenced state for a period of time.

Informational spin fields have the ability to affect matter under certain circumstances, especially in materials undergoing a phase change, and tend to influence the alignment of electron, nuclear, and atomic spins. This fact was verified by experiments carried out in the Soviet Union using the Mossbauer Effect. In this effect, the only known interaction with the material under investigation is through spin, and the ISF created by devices designed by Anatoly Akimov did affect the materials. Thus, it was proven that these informational fields relate to spin, which is why the term “spin” is being included in the name of these informational fields described herein.

It is presently postulated by some scientists that ISFs carry information, and can impart that information to matter in the form of phase information associated with varying degrees in the precession of spins. Experiments by Dr. Akimov and others show that ISFs can, under certain circumstances, affect crystal structure and molecular structure, and consequently physical properties, in materials.

Informational spin fields are known to be generated in numerous ways. Statically generated ISFs occur inherently with physical geometry. For example, stationary objects, such as spheres, cones, cylinders, and tetrahedrons, all generate static ISFs. The intensity of static ISFs increases with specific ratios in the geometry of the object, such as, for example, the phi ratio of approximately 1.618, as well as with the increasing size of the object.

Dynamically generated ISFs are produced by bodies with angular motion, for example, rotating spheres and nuclear and atomic particles. Dynamically generated ISFs are produced by electromagnetic radiation as well, such as by light and by rotating magnetic fields. An example of an ISF created when rotating a magnet about an axis is illustrated in a device presently produced in Kazakhstan and marketed internationally by Alexander A. Shpilman. Dynamically generated ISFs can also be produced by combinations of geometry and changing electromagnetic fields. Soviet patent No. 1748662 patenting such a device together with its use in modifying the properties of materials was issued in 1992 with priority since 1990 to Anatoly Akimov et al.

The existence of biofields surrounding living beings has been established by scientists over the past several decades. Valerie Hunt, a Professor Emeritus of UCLA, was able use the patterns in electromyograph signals to consistently correlate patterns in the human biofield observed by individuals who could directly perceive them. The results of her 25 years of research and clinical studies demonstrating these results were presented in 1989 in her book, Infinite Mind. More recently, Konstantin Korotkov, Professor of Physics at St. Petersburg State Technical University in Russia, introduced a commercial device using a Gas Discharge Visualization (GDV) technique (Kirlian method), and is also able to correlate parameters measured by that device with those patterns observed by individuals who could directly perceive the human biofield. The GDV device outputs have successfully correlated with the real time introduction of stimulation to human subjects experiencing aromas, physical injury, and other stimuli. Biofields themselves appear to be informational spin fields, based upon research observations of Dr. Anatoly Akimov correlating images of biofields observed by individuals who could directly perceive them, with their direct perceptions of the outputs of dynamic ISF generators.

### BRIEF SUMMARY OF THE INVENTION

The present invention mitigates stress in individuals and improves the efficiency of stress alleviation afforded by other available environments and techniques. The present invention provides a device, system, apparatus and method for reducing stress in an individual by creating an enhanced informational spin field environment substantially surrounding the individual. The term “informational spin field” or “ISF,” as used herein, refers to a field also commonly known as a torsion field.

In embodiments of the present invention, an apparatus for destressing is provided that is configured to temporarily accommodate an individual, preferably such that the individual can assume a resting position. The apparatus is configured with a series of elements whose geometrical arrangement corresponds to a predetermined pattern. In one example, the apparatus comprises a support structure configured to act as a bed and two end structures joined to and orthogonal to the bed. In one embodiment of the present invention, the end structures each comprise multiple layers of electrically insulating material, such as wood. In embodiments of the present invention, a pattern of metallic tape, such as copper, is laminated between each wood layers comprising each end structure. In one embodiment of the present invention, the pattern of the copper tape comprises a six-pointed star geometry comprised of two superposed equilateral triangles, also know as a Star of David. Preferably, the patterns are arranged opposite each other such that each apex of a star pattern can be connected to a corresponding apex by a conductive member that is mutually orthogonal to both end structures. In one embodiment of the present invention, each end structure comprises a trilayer assemblage of wood layers, in which the middle layer includes a metallic tape pattern affixed thereto.

In one embodiment of the present invention, the end members are joined to each other by a series of six metallic tubes that are substantially orthogonal to each of the end members. Preferably, the orthogonal metallic tubes are mutually arranged to each interconnect a point of a metallic Star of David that is laminated between outer and inner boards of an end structure with a corresponding point in a similar structure on the opposite end member. The laminated Star of David pattern may be affixed to a middle board of trilayer structure, or alternatively may be at the interface of an inner and outer board of a bilayer end structure. Preferably, six metallic tubes are arranged to interconnect all six apices of a metallic Star pattern located in a first end structure with a corresponding six apices in a metallic Star pattern located in the opposite end structure to the first end structure. Accordingly, an individual resting on a bed disposed between the end structures lies within a hexagonal prism whose long edges parallel to the cylinder axis are defined by the metallic pipes.

In embodiments of the present invention, the apparatus for destressing further includes a hexagonal projector located in an upper portion of the apparatus and configured with a series of six cones. Preferably, the hexagonal projector is configured to slide in a direction parallel to the bed structure. Preferably, the destressing apparatus also includes a ball radiator that includes a small metallic sphere that is configured to slide in a direction parallel to that of the hexagonal projector.

In accordance with the above-described elements, a static informational spin field environment can be provided in a spatial region designed to accommodate individuals of varying size within the destressing apparatus. Once substantially inside a region corresponding to the hexagonal prism, the static ISF environment created by the destressing apparatus efficiently interacts with the biofield of the individual, such that a destressing process is initiated.

In other embodiments of the present invention, the disclosed device, system, apparatus and method provide an informational spin field environment substantially surrounding an individual which is at least partially derived from one or more dynamically produced informational spin fields, wherein electromagnetic components associated with producing one or more of such dynamically produced informational spin fields have first been substantially separated therefrom, such one or more dynamically produced informational spin fields being then conducted to the environment substantially surrounding the individual. The term “dynamically produced informational spin field,” as used herein, refers to an ISF that is produced at least in part from the time-dependent variation of an entity, such as a varying magnetically-induced spin field, electromagnetic signal, electromagnetic current, or electromagnetic radiation.

In embodiments of the present invention, an apparatus is configured to establish an ISF environment in a region configured to accommodate a resting individual, wherein the ISF environment comprises a dynamically produced informational spin field resulting predominantly or in whole from inputs from a magnetic, electric, or electromagnetic source. In other embodiments of the present invention, the ISF environment is created by a combination of elements configured to generate static ISFs together with sources that serve to generate one or more dynamic ISFs, such as electromagnetic, magnetic, or electrical signals.

The present invention provides a system for alleviating or reducing stress in an individual comprising an informational spin field environment substantially surrounding an individual, which is at least partially derived from one or more dynamically produced informational spin fields, wherein electromagnetic components associated with producing one or more of such dynamically produced informational spin fields have first been substantially blocked from propagating with the informational spin field produced therefrom, such one or more dynamically produced informational spin fields being then conducted without any accompanying electromagnetic field to the environment substantially surrounding the individual.

The present invention also provides a method for alleviating or reducing stress in an individual, comprising providing an informational spin field environment substantially surrounding such individual, which is at least partially derived from one or more dynamically produced informational spin fields, wherein electromagnetic components associated with producing one or more of such dynamically produced informational spin fields have first been substantially separated blocked from propagating with the informational spin field produced therefrom, such one or more dynamically produced informational spin fields being then conducted without any accompanying electromagnetic field to the environment substantially surrounding the individual.

In one embodiment of the present invention, the dynamically produced informational spin field source utilizes an electromagnetic signal to generate an informational spin field, wherein the electromagnetic signal itself is substantially separated from the informational spin field produced therefrom, said informational spin field produced therefrom being then conducted to the environment substantially surrounding the individual. In one embodiment of the present invention, the dynamic informational spin field source utilizes an electromagnetic signal derived from a musical sound input to generate an informational spin field, wherein the electromagnetic signal itself is substantially blocked from propagating with the informational spin field produced therefrom, said informational spin field produced therefrom being then conducted without any accompanying electromagnetic field to the environment substantially surrounding the individual.

In one embodiment of the present invention, the dynamic informational spin field source utilizes an electrical signal from a compact disk (CD) or magnetic tape player to generate an informational spin field, wherein the electromagnetic components of the electrical signal itself are substantially blocked from propagating along with the informational spin field produced therefrom, wherein the informational spin field produced therefrom is conducted to the environment substantially surrounding the individual without accompanying electromagnetic radiation or electric signals.

In one embodiment of the present invention, the informational spin field environment is at least partially derived from one or more light sources wherein the electromagnetic components of the light emitted therefrom are substantially blocked from propagating with the informational spin field produced by the light, said informational spin field being then conducted without accompanying light to the environment substantially surrounding the individual.

In one embodiment of the present invention, means are provided to modify and/or adjust the informational spin field environment in either composition or intensity or both so as to be made harmonious for an individual substantially surrounded by such environment. In one embodiment, the informational spin field of the present invention is modified and/or adjusted in either composition or intensity or both in response to one or more autonomic responses of the individual substantially surrounded by said informational spin field environment so as to make it harmonious for said individual.

In one embodiment of the present invention, the informational spin field environment is partially derived from one or more statically generated informational spin fields.

In one embodiment of the present invention, means are provided to cause the primary localization of the information spin field environment within the vicinity of the individual.

In one embodiment of the present invention, either music or light is additionally provided to the individual directly in order to provide an aesthetic benefit.

The present invention offers the potential of improved efficiency as compared to means of achieving stress reduction by the practices of the prior art. Significantly positive results are observable in 15 to 30 minutes exposure to the informational spin field environment of the present invention. In a society in which the time to deal with one's own needs is frequently scarce, this advantage of the present invention is very important. Moreover, this feature offers the possibility for commercial employers to provide the benefit of such a device to employees in the work environment to improve morale and productivity, since the economic return in terms of increased worker efficiency does not have to be very large to justify perhaps only a 15-minute break exposure to the environment of the present invention.

While not wishing to be bound by any particular theory, it is believed that all destressing environments owe their effects to the presence of ISFs. Unlike the case with music and light healing environments, in which the inputs to such environments are acoustic and electromagnetic, any ISF intensity of such environments must be limited to lower levels because of potential discomfort or even harm to an individual at high levels of sound or light exposure. By virtue of the ability to prevent electromagnetic and acoustic signals from propagating in the environment surrounding an individual, the present invention provides a means to achieve higher ISF levels in the immediate physical surroundings of an individual without the need to incur high levels of electromagnetic radiation or acoustic signals in the same physical surroundings. This facilitates optimizing the destressing effect within a minimum of time without introducing unwanted or negative side effects of excessive electromagnetic or acoustic energy near the individual. Moreover, acoustic or electromagnetic components can in themselves create unwanted interactions in certain instances. The feature of the present invention of minimizing or eliminating any acoustic inputs and filtering out electromagnetic components from ISF inputs to the environment substantially surrounding the individual permits the creation of effects on the individual that are solely positive, and therefore adds to the efficiency of achieving the destressing result.

Unlike certain therapies, such as spas, hot tubs and saunas, which produce relaxation and stress alleviation at the expense of creating lethargy, individuals exposed to the informational spin field environment of the present invention report feeling energized, yet relaxed.

### BRIEF DESCRIPTION OF THE DRAWINGS

In order that the invention will be readily understood, a more particular description of the invention will be rendered by reference to specific embodiments that are illustrated in the appended drawings. Understanding that these drawings depict only typical embodiments of the invention and are not therefore to be considered limiting of its scope, the invention will be described and explained with additional specificity and detail through the use of the accompanying figures, in which:

FIG. 1 depicts one embodiment of an apparatus in accordance with the present invention, in which an individual is situated in a reclining position appropriate to its use;

FIG. 2a is a schematic perspective view of a system for destressing, in accordance with an embodiment of the present invention;

FIG. 2b is a schematic diagram of a perspective view of a hexagonal prism region defined by longitudinal members, in accordance with one embodiment of the present invention;

FIG. 2c is a schematic diagram of a front view and side view of an end structure of the system of FIG. 2a, in accordance with one embodiment of the present invention;

FIG. 2d is a schematic diagram of an exploded view of the end structure of FIG. 2c, in accordance with one embodiment of the present invention;

FIG. 2e is a schematic diagram that illustrates an exemplary hexagonal metallic tape pattern and collector of the middle panel of the end structure of FIG. 2c, in accordance with an embodiment of the present invention;

FIG. 2f is a schematic diagram that illustrates the opposite side of the panel shown in FIG. 2e, showing details of the collector, according to an embodiment of the present invention;

FIG. 2g is a schematic diagram that illustrates details of a collector star plate, in accordance with one embodiment of the present invention;

FIG. 2h is a schematic diagram of a side view of the destressing system of FIG. 2a, showing the collector in relation to the system;

FIG. 2i is a schematic diagram that depicts details of an inner panel of the end structure of FIG. 2c, in accordance with an embodiment of the present invention;

FIG. 2j is a schematic diagram that depicts a side view of an end structure of FIG. 2c, showing carriage bolt locations, in accordance with an embodiment of the present invention;

FIG. 2k is a schematic perspective view of a system for destressing, in accordance with another embodiment of the present invention;

FIG. 2l is a schematic illustration that depicts elements of a system for destressing in accordance with an embodiment of the present invention;

FIG. 2m is a schematic illustration that depicts elements of a system for destressing in accordance with an embodiment of the present invention;

FIG. 3 is a schematic diagram that illustrates details of an end structure outer panel, in accordance with one embodiment of the present invention;

FIG. 4 is a schematic diagram that shows the configuration and dimensions of the two inner end panels of the system illustrated in FIG. 2k, with the copper tape shown in FIG. 4 applied to one side of each of the two inner end panels on the faces respectively away from the individual as they would recline within the apparatus, and with the slot shown situated on the edge of the respective inner end panel that would be on the reclining individual's left, permitting one copper tube on that side to be removed to permit convenient ingress and egress of the individual from the apparatus, according to an embodiment of the present invention;

FIG. 5 is a schematic diagram of an assembly drawing for a bed of the system illustrated in FIG. 2a, upon which the individual is shown reclining in FIG. 1;

FIG. 6 is a schematic diagram of an assembly drawing for a top assembly configured for use in the system illustrated in FIG. 2a;

FIG. 7a is a schematic diagram of an assembly drawing for a foot assembly configured for use with the apparatus depicted in FIG. 2a, according to an embodiment of the present invention;

FIG. 7b is a schematic diagram of an assembly drawing for a foot assembly configured for use with the apparatus depicted in FIG. 2k, according to another embodiment of the present invention;

FIG. 8a is a schematic diagram of an assembly drawing of an exemplary dynamic ISF generator component configured for use with the apparatus depicted in FIG. 2a, according to an embodiment of the present invention;

FIG. 8b is a schematic diagram of an assembly drawing of another exemplary dynamic ISF generator component configured for use with the apparatus depicted in FIG. 2k, according to an embodiment of the present invention;

FIG. 9 is a schematic diagram of a diagram showing detail of electrical connections for the capacitor component of the ISF generators shown in FIGS. 8a and 8b, according to an embodiment of the present invention;

FIG. 10 is a schematic diagram showing an exemplary configuration and exemplary dimensions of the copper cone component of the ISF generators of FIGS. 8a and 8b, according to an embodiment of the present invention;

FIG. 11 is a schematic diagram showing an exemplary configuration and exemplary dimensions of the bottom Teflon™ cone mount component of the ISF generator of FIGS. 8a and 8b, according to an embodiment of the present invention;

FIG. 12 is a schematic diagram showing an exemplary configuration and exemplary dimensions of the top Teflon™ cone mount component of the ISF generator of FIGS. 8a and 8b, according to an embodiment of the present invention;

FIG. 13 is a schematic diagram showing an exemplary configuration and exemplary dimensions of the ring magnet component of the ISF generator of FIGS. 8a and 8b, according to an embodiment of the present invention;

FIG. 14 is a schematic diagram showing an exemplary configuration and exemplary dimensions of the bronze Teflon™ capacitor component of the ISF generator of FIGS. 8a and 8b, according to an embodiment of the present invention;

FIG. 15 is a schematic diagram showing an exemplary configuration and exemplary dimensions of a metal housing component of the ISF generator of FIG. 8b, according to an embodiment of the present invention;

FIG. 16 is a schematic diagram of an assembly drawing of an exemplary fiber coupler assembly component configured for use generally with the system depicted in FIG. 27a, according to an embodiment of the present invention;

FIG. 17 is a schematic diagram showing an exemplary configuration and exemplary dimensions of the insulator cone components of the fiber coupler assembly of FIG. 16, according to an embodiment of the present invention;

FIG. 18a is a schematic diagram of an assembly drawing in cross-section of an exemplary lamp ISF generator assembly of the system depicted in FIG. 27a, according to an embodiment of the present invention;

FIG. 18b is a schematic diagram of a perspective view, top view, side view, and bottom view of the lamp reflector component of FIG. 18a, according to an embodiment of the present invention;

FIG. 18c is a schematic diagram of perspective views and a side view of the lamp reflector and lamp box of the exemplary lamp ISF generator assembly of FIG. 18a, according to an embodiment of the present invention;

FIG. 19 is a schematic diagram of an assembly drawing of a hexagonal projector base structure, in accordance with one embodiment of the present invention;

FIG. 20a is a schematic diagram of an assembly drawing showing an exemplary configuration and exemplary dimensions of an exemplary ISF projector comprising a copper cone assembly, in accordance with an embodiment of the present invention;

FIG. 20b illustrates an example of a copper or phosphor bronze cone, arranged in accordance with one embodiment of the present invention;

FIG. 21a is a schematic diagram of an assembly drawing of an exemplary hexagonal cone projector configured for use in the apparatus depicted in FIGS. 2a and 27a, according to an embodiment of the present invention;

FIG. 21b is a schematic diagram of a hexagonal distributor component of the apparatus of FIG. 21a, according to an embodiment of the present invention;

FIG. 21c is a schematic diagram of an assembly drawing of a housing for the apparatus of FIG. 21a, according to an embodiment of the present invention;

FIG. 21d is a schematic diagram of an assembly drawing of a cover for the apparatus of FIG. 21a, according to an embodiment of the present invention;

FIG. 22a is a schematic diagram of a ball radiator assembly in accordance with an embodiment of the present invention;

FIG. 22b is a schematic diagram of the configuration of a ball radiator assembly, infrared (IR) camera, IR light source, and hexagonal projector assembly in an apparatus, such as that depicted in FIG. 1, in accordance with an embodiment of the present invention;

FIG. 23 is a schematic depiction of a ball radiator assembly in accordance with an embodiment of the present invention;

FIG. 24 is a schematic diagram that shows exemplary details of the hexagonal projector, infrared (IR) camera, IR light source, ball ISF radiator assembly, and mattress components with respect to their relative positions in the apparatus depicted in FIG. 2k, in accordance with an embodiment of the present invention;

FIG. 25 is a schematic diagram of an assembly drawing of an exemplary distribution assembly of the apparatus depicted in FIG. 21a, according to an embodiment of the present invention;

FIG. 26 is a schematic diagram that details exemplary connections between two copper panels of the foot assembly of FIG. 7b, the distribution assembly of FIG. 25, and the copper tape of FIG. 4 at its junctions with the copper tubes, according to an embodiment of the present invention;

FIG. 27a is a circuit diagram of exemplary connections and electromagnetic currents and their resulting ISF flows that are being input into a destressing apparatus, in accordance with an embodiment of the present invention;

FIG. 27b illustrates a wiring diagram for a system used to supply a signal derived from a music player to an ISF generator such as that shown in the embodiments of FIGS. 8a and 8b, according to an embodiment of the present invention;

### DETAILED DESCRIPTION OF THE INVENTION

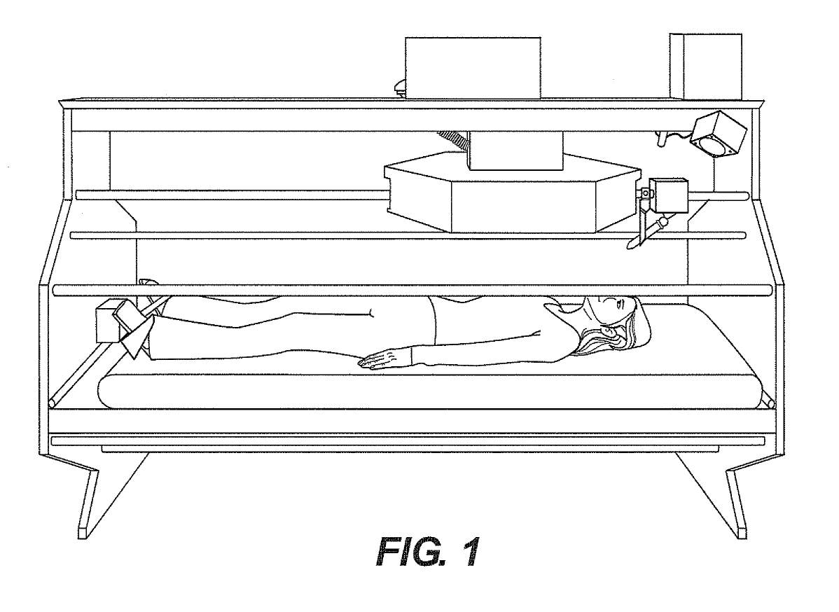

While not wishing to be bound by any particular theory, it appears that the device, system, apparatus and method of the present invention results in an ISF flow that circulates in the informational spin field environment substantially surrounding an individual. Such flow does not appear to require direct contact with the individual substantially within such environment in order to occur. For example, although a foot paddle is provided in one embodiment of the present invention depicted in FIG. 1, contact of the individual with the foot paddle does not appear to be required, although the flows appear somewhat more intensive when contact of the feet with the paddles is employed, with or without socks, as reported by individuals experiencing the environment, based on their perceptions.

One aspect of the device, system, apparatus and method of the present invention provides means for enhancing an informational spin field environment substantially surrounding the individual. Biofields themselves appear to be informational spin fields surrounding all living beings. The present invention reduces stress in an individual by creating an enhanced informational spin field environment substantially surrounding the individual. This facilitates the process in which an individual changes his or her own biofield in a manner that serves to reduce stress.

In particular, in an embodiment of the device, system, apparatus and method of the present invention, the dynamic ISF input or inputs are provided in a manner in which they are harmonious to the individual at the time of the individual's presence substantially within the enhanced informational spin field environment.

FIG. 1 depicts one embodiment of an apparatus in accordance with the present invention. In that embodiment, copper tubes and copper tape are provided in a geometric configuration that localizes an ISF environment substantially surrounding the individual within the main support assembly thereof.

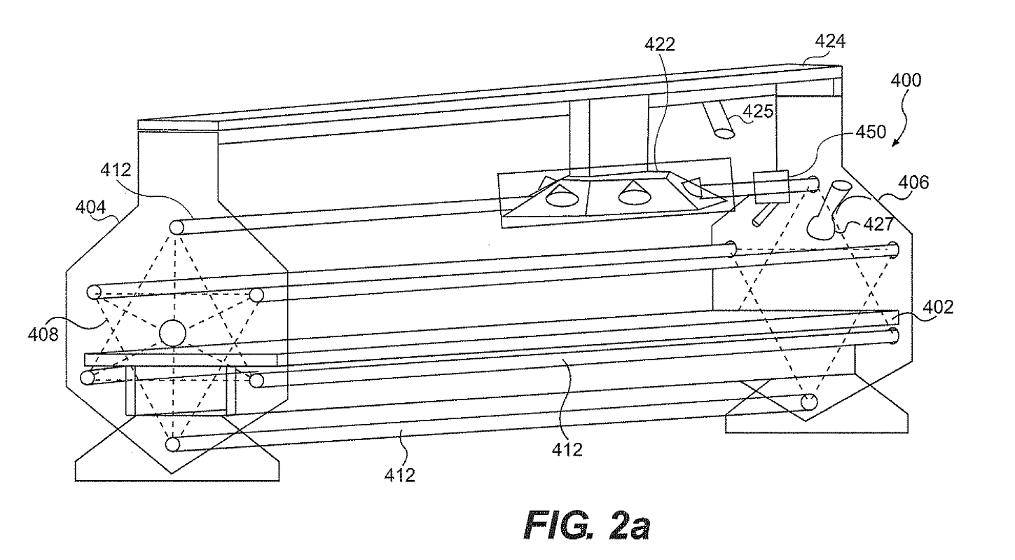

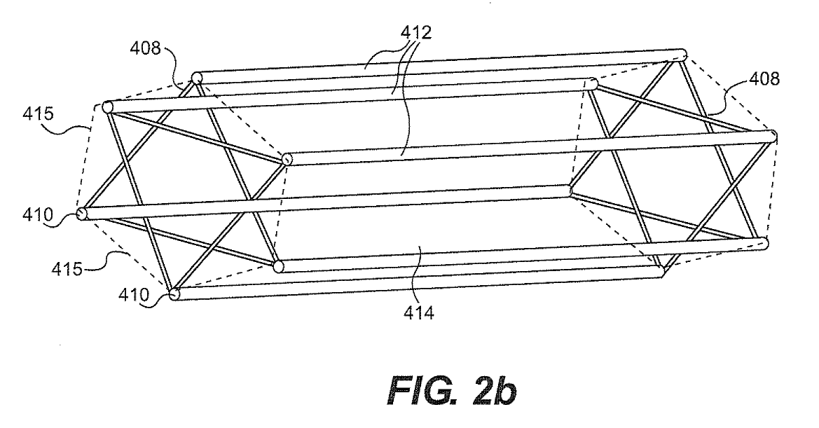

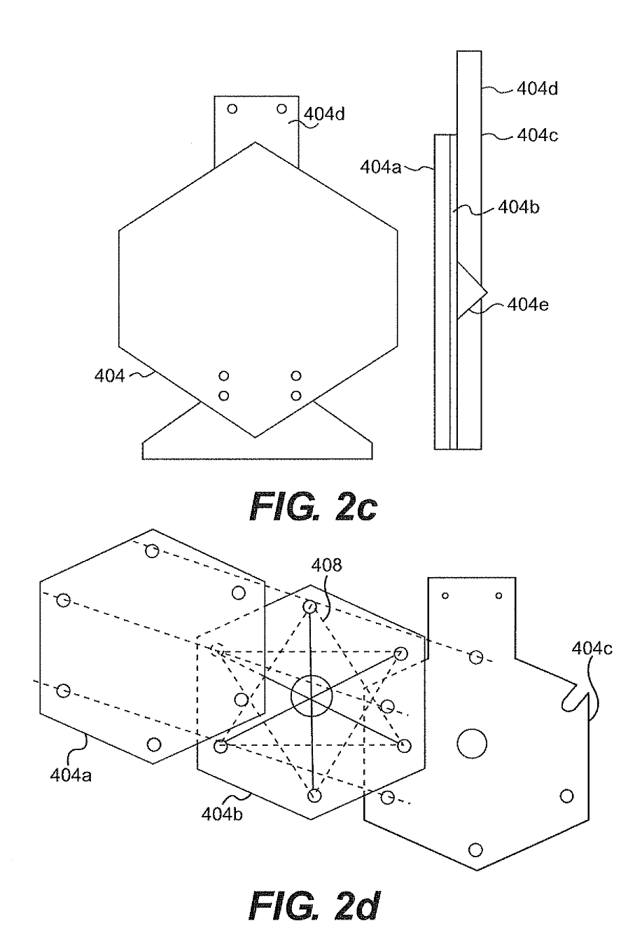

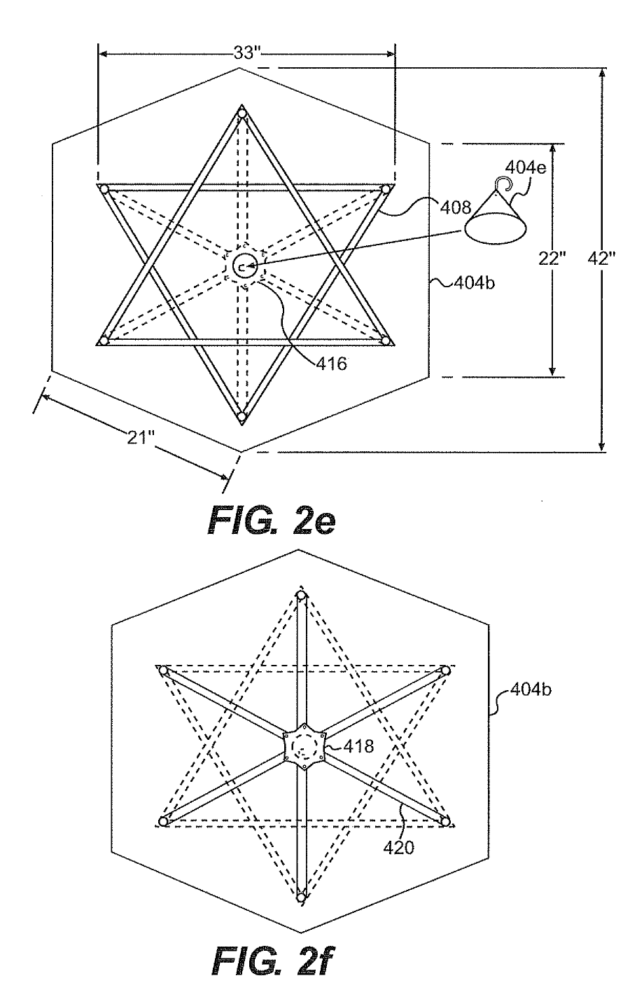

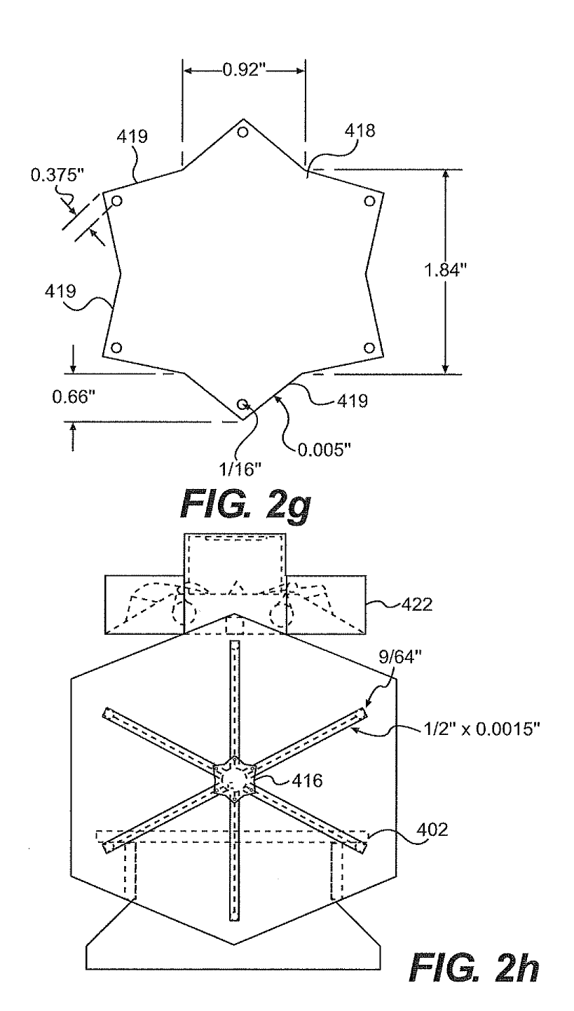

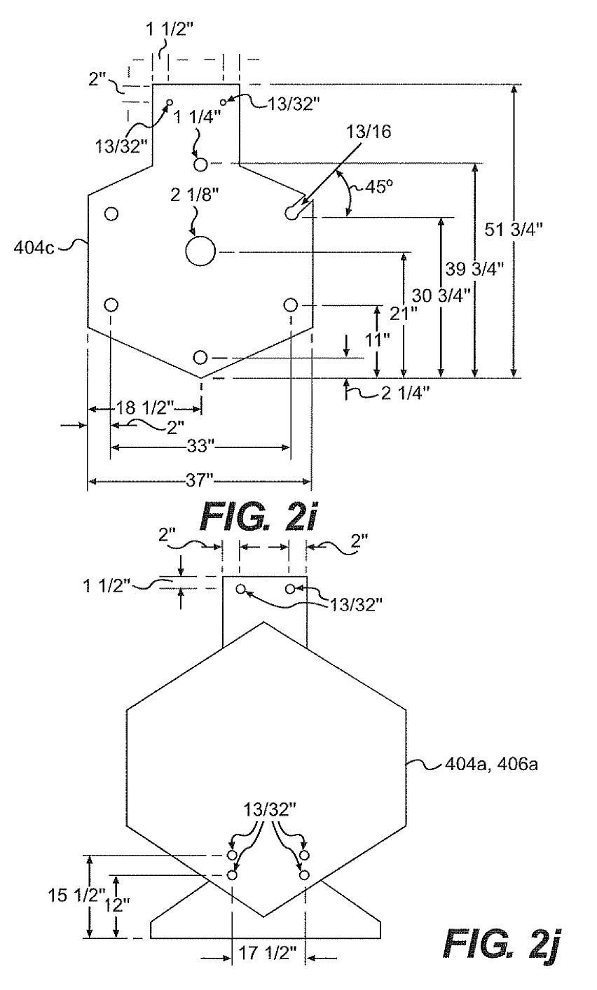

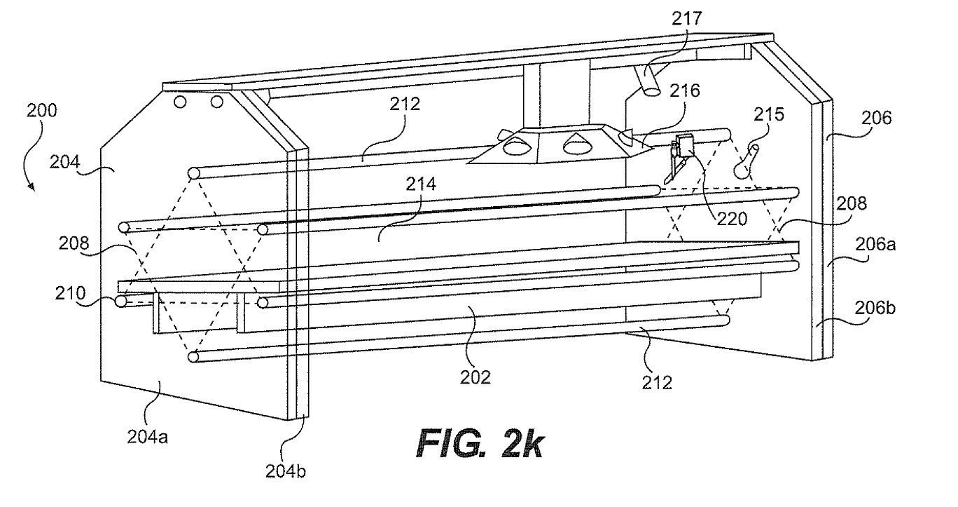

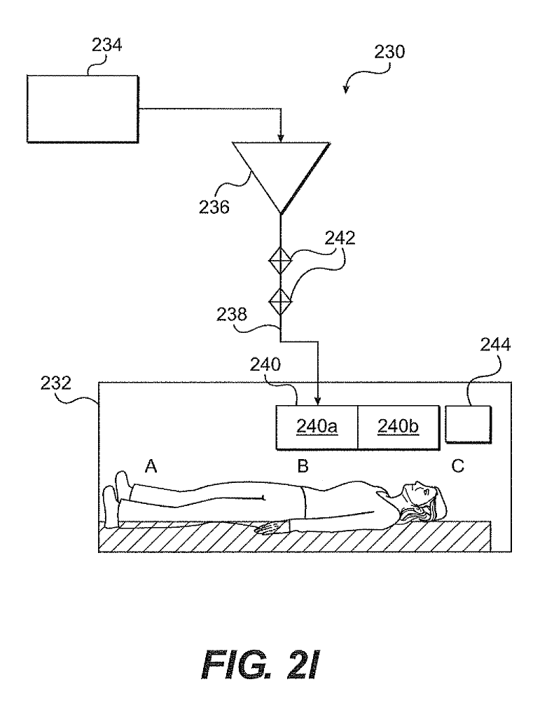

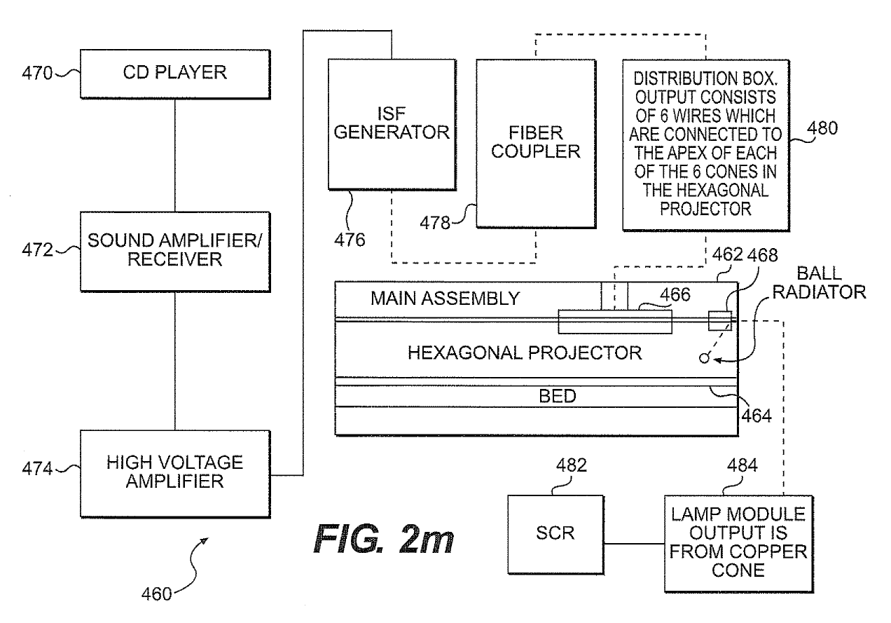

FIG. 2a is a schematic perspective view of a system 400 for destressing, in accordance with an embodiment of the present invention. System 400 includes a bed 402 that is affixed at each end to end members 404 and 406.

As depicted in FIG. 2d, end members 404 and 406 each comprise a trilayer structure, 404a, 404b, 404c and 406a, 406b, 406c respectively, which preferably comprises a wood-based material. Sandwiched between outer layer 404a (or 406a) and inner layer 404c (or 406c) is a layer 406b that preferably includes a wood substrate to which is affixed a star pattern 408, preferably comprising metallic tape such as copper tape (e.g., ½ inch wide and 0.0015 inches thick, with self-adhesive). Star pattern 408 comprises a pair of overlapping and oppositely facing triangles each approximately equilateral and arranged so that the six corners of the overlapping triangles form the points of a regular hexagon having equal sides. Such a star pattern is commonly referred to as a Star of David pattern. In the example shown, each of the six star apices 410 is connected to a longitudinal member (tube) 412 (see FIG. 2a) that in turn connects that apex with a corresponding apex in the opposite star pattern. The longitudinal members are designed to conduct ISF fields and can comprise a solid metal, insulator, or other material. Longitudinal members 412, also referred to as tubes, may be, for example, solid bars, hollow cylinders, or cylinders containing solid inserts. Preferably, each tube 412 comprises an outer metal tube (not separately shown), such as copper and further includes a supporting wooden dowel (not shown) within the metal tube. The tubes 412 are arranged to be substantially orthogonal to the planes of end members 404 and 406. Thus, tubes 412 are mutually arranged in a hexagonal array as viewed along the axis of the tubes. This arrangement serves to define a larger hexagonal prism space 414, as illustrated in FIG. 2b. The base edges 415 of the prism space are defined by connecting adjacent star apices 410 and are all identical in length. The length of the prism space is equivalent to the physical separation of opposing star surfaces whose apices are the points of each prism base. The height and relative lateral position of bed 402 is configured such that an individual lying on bed 402 is substantially or wholly within the space defined by prism space 414, as illustrated in FIG. 2a.

The metallic tubes 412, together with metallic tape patterns 408 are configured to establish and direct an ISF environment particularly within the region defined by prism space 414, although ISFs can extend into the region outside of prism space 414. In one embodiment of the present invention, the metallic tube length between opposing star surfaces embedded within members 404 and 406 is 88 inches.

FIG. 2c illustrates details of end structure 404, in accordance with one embodiment of the present invention. In one embodiment of the present invention, each of layers 404a, 404b, and 404c have the shape of a hexagon, save for protrusion 404d on the top portion of 404c. Layer 404c also has an opening that accommodates cone structure 404e (discussed further below) that is configured to direct ISFs into the foot area of an individual lying on bed 402. End structure 406 is preferably configured substantially the same as end structure 404, except that cone structure 404e is not present in end structure 406.

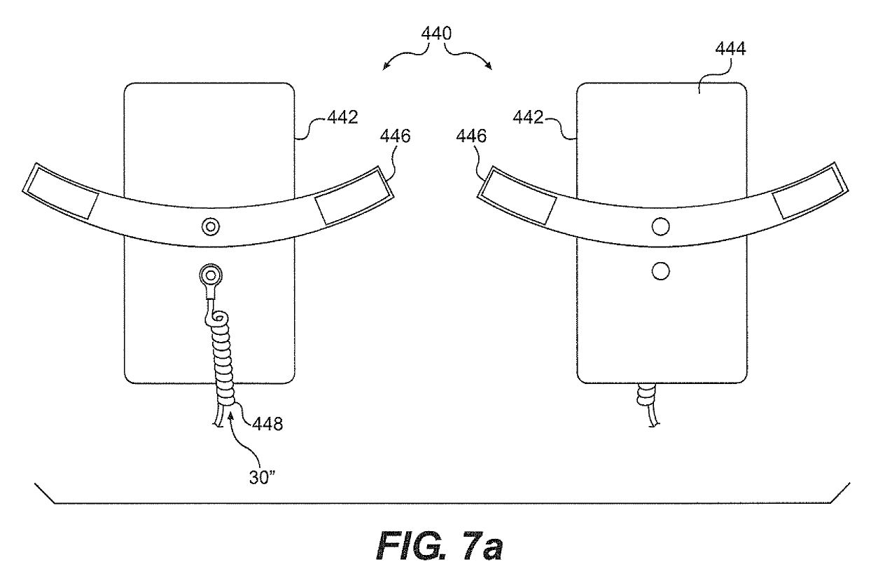

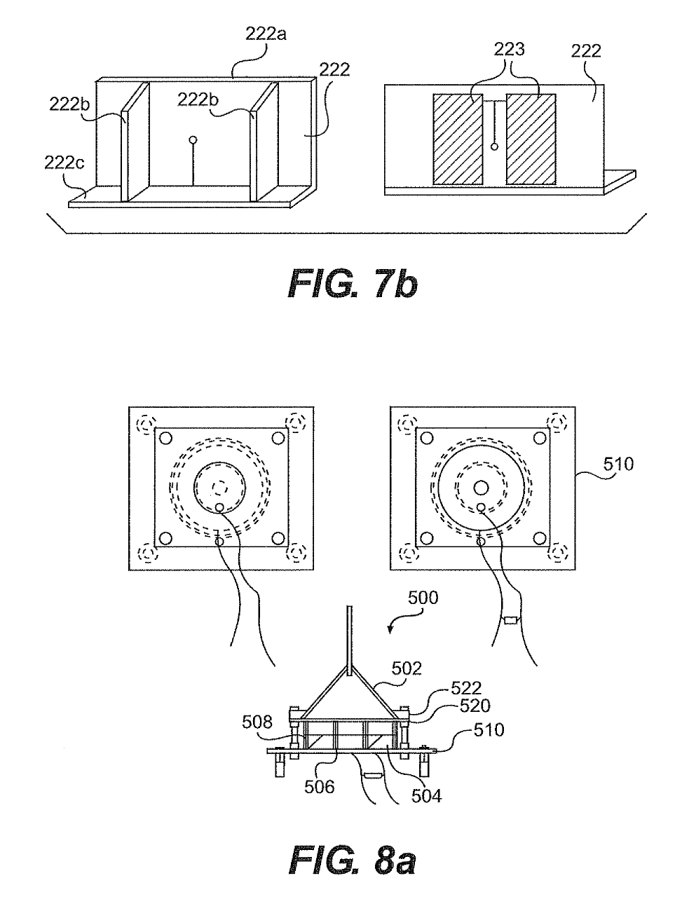

FIG. 2e illustrates details of a star shaped metallic tape pattern 408 and collector 416, in accordance with an embodiment of the present invention. Star pattern 408 is affixed to panel 404b on a surface that is inwardly disposed toward the region where an individual lies on bed 402. Collector 416 comprises cone structure 404e and star plate 418, depicted more clearly in FIG. 2f. Cone 404e is configured to couple to a foot panel or foot paddles, described in more detail with respect to FIGS. 7a and 7b. Cone 404e is affixed to star plate 418, which in turn is affixed to radial pattern 420. As depicted in FIG. 2f, pattern 420 is affixed to the side of panel 404b opposite to that which star pattern 408 is affixed, and preferably comprises metallic tape similar to or the same as that used for star pattern 408. Thus, collector 416 serves to collect ISF fields that are distributed along metallic tubes 412 and direct them to cone 404e, which itself is configured to couple to foot panels or foot paddles to direct fields into the lower extremities of an individual lying on bed 402.

In one embodiment of the present invention, the distance from opposite points on hexagonal panels 404a, 404b, and 404c is 42 inches, the length of a vertical panel side is 22 inches, the length of non-vertical panel sides is 21 inches, the distance between next nearest neighboring apices in star pattern 408 (i.e., the distance between two vertices of one of the two large triangles that make up the star pattern) is 33 inches, and the width of panels 404a, 404b, and 404c is 37 inches. The 33″ alternate point to point dimension of the stars, as well as the 88″ dimension between opposing star surfaces are preferred dimensions.

FIG. 2g illustrates details of collector star plate 418, in accordance with one embodiment of the present invention. Star plate 418 preferably comprises a regular hexagon whose sides each define the base of a phi ratio triangle. In one example, the base of the phi ratio triangle is 0.92 inches, the height is 0.66 inches, and the metal thickness is 0.005 inches (e.g., annealed copper sheet). Holes (e.g., approximately 1/16 inch in diameter) are provided about 0.375 inches from each triangle apex to provide openings for a fastener (not shown) to fasten the plate to members of radial pattern 420.

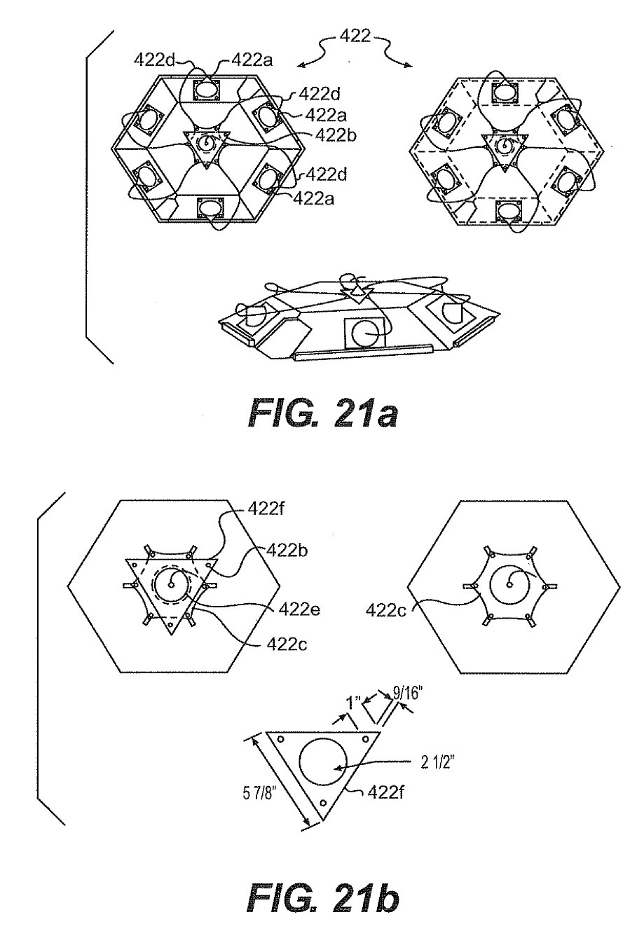

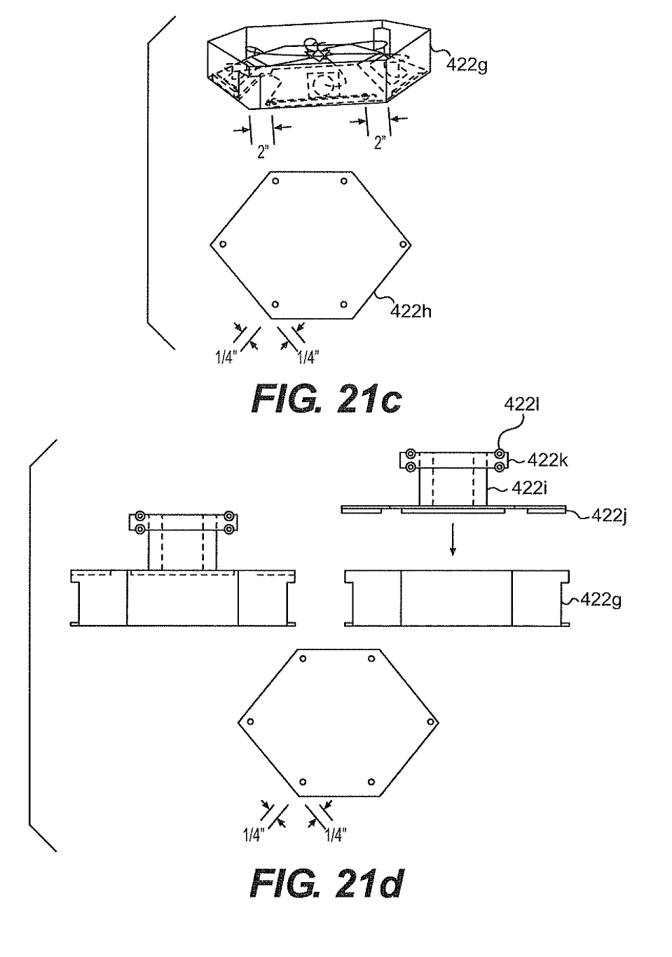

As depicted in FIG. 2h, collector 416 is disposed above bed 402 and in the center of hexagonal prism 414. Also shown in FIG. 2h is projector 422 which is configured to project ISFs from a top region of apparatus 402 (see also FIG. 2a), as discussed below with respect to FIGS. 21a-d. FIG. 2h also shows how the metallic tape connects to the six tubes. In one embodiment, the tubes are copper tubes, the ends of which are disposed in copper cap sockets (e.g., 1 inch copper sweat caps) whose bases are secured (e.g., riveted) to the middle panel (e.g., panel 404b) of the end structure.

FIG. 2i depicts details of inner panel 404c in accordance with an embodiment of the present invention. As shown, panel 404c is configured with six 1.25 inch diameter holes to accommodate metal tubes 412. Panel 404c also includes a central hole having a 2.125 inch diameter that is used to clamp cone structure 404e to star plate 418 when panels 404c and 404b are joined together. Panel 404c also includes a slot to one of the six 1.25 inch diameter holes so that a tube 412 can be removed and replaced, to permit ingress and egress by an individual.

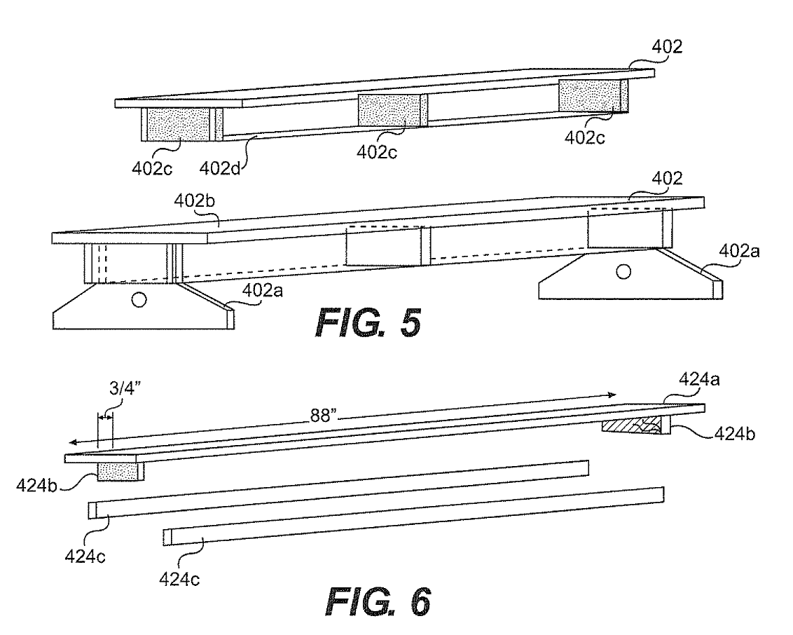

FIG. 2j depicts details of an outer panel, which can be outer panel 404a or 406a, in accordance with one embodiment of the present invention. Preferably, a pattern of four carriage bolt holes is provided in a lower portion of panel 404a to allow bolting to bed 402, discussed further below with respect to FIG. 5, while a pattern of two bolt holes is provided in a top portion of panel 404c for fastening to a top assembly 424 (see FIG. 2a), discussed further below with respect to FIG. 6.

FIG. 5 illustrates details of bed 402, in accordance with one embodiment of the present invention. Bed 402 includes feet 402a and horizontal member 402b. Bed 402 is supported by cross braces 402c and long braces 402d. Bed 402 is configured to be flush with the inner panels 404c and 406c when assembled into system 400. Accordingly, end structures 404 and 406 are supported by feet 402a, as illustrated in FIG. 2a and 2j. The outer cross braces 402c bolt to the end structures via the carriage bolt locations shown in FIG. 2j. Bed 402 can be made of, for example, birch plywood. Although bed 402 is shown as containing legs, as an alternative, the bed could be supported by the end members, as shown for bed 202 of FIG. 2k.

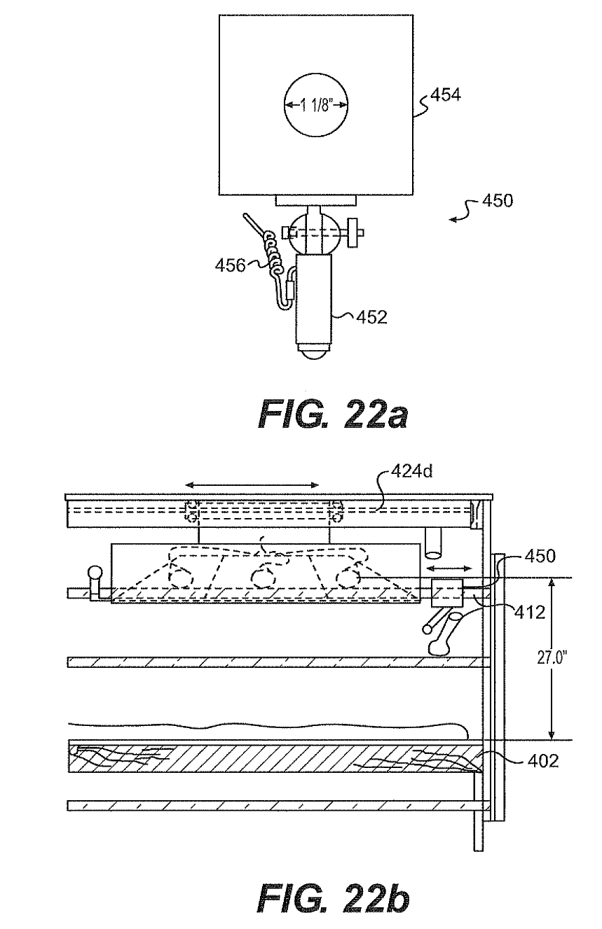

FIG. 6 illustrates further details of top assembly 424, in accordance with one embodiment of the present invention. Assembly 424 includes top surface 424a, cross braces 424b, and side pieces 424c. In one embodiment, as shown in FIG. 22b, side pieces 424c each include an aluminum track 424d that acts to guide projector 422 in a horizontal plane.

FIGS. 7a illustrates a foot assembly 440 that can be coupled to collector 416, in accordance with one embodiment of the present invention. Foot assembly 440 comprises a pair of foot paddles 442 that preferably include a silver sheet on front surface 444. Feet can be secured to the paddles with fasteners 446 (e.g., a strap with a hook and loop fastener). Cord 448, disposed on the back of foot assembly 440, couples paddles 442 to a collector, such as collector 416 illustrated above. Referring again to FIG. 2a, paddles 442 are configured so that an ISF established along metallic tubes 412 can be conducted to the lower extremity region of an individual in bed 402.

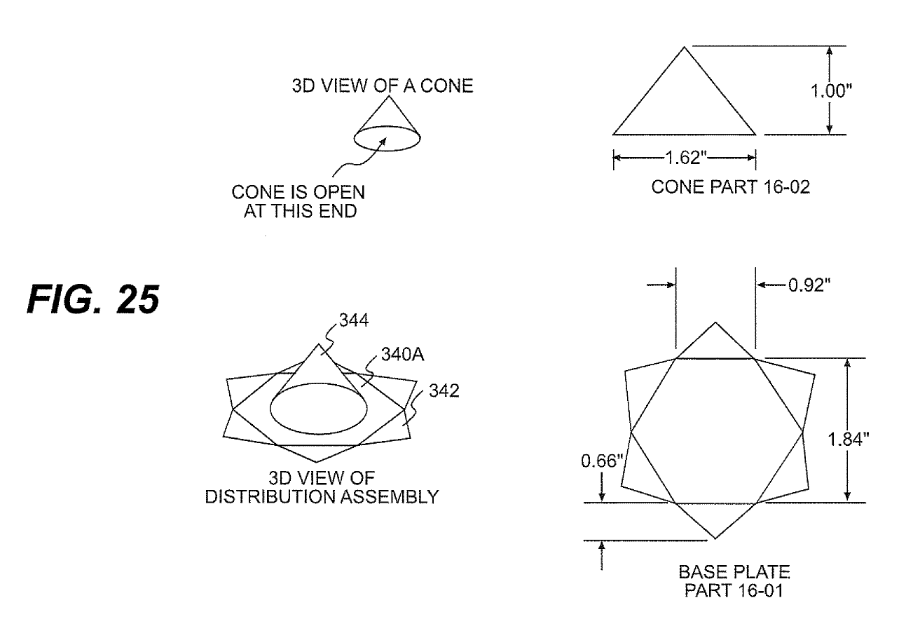

As illustrated in FIG. 2a, and further in FIG. 21a, system 400 also includes a hexagonal assembly (projector) 422, which can be used to provide an ISF environment inside structure 402. In one embodiment of the present invention illustrated in FIG. 21a, hexagonal projector 422 is configured with a series of six cylindrical cones 422a, preferably mutually arranged so that their cone axes all converge upon a point. Preferably, cones 422a are phi ratio cones in which the ratio of base to height is about 1.618. In one embodiment, illustrated in FIG. 2a, the hexagonal assembly 422 is configured such that the axes of all cones converge at a point (not shown) above bed 402. In embodiments of the present invention, discussed further below, hexagonal projector 422 is slidable in the longitudinal direction of tubes 412, such that the convergence point can be positioned above a specific region, such as the heart chakra of an individual resting on bed 402. The arrangement of cones 422a is such that the base of each cone is downwardly disposed (closer to bed 402) with respect to the cone apex. Accordingly, any static ISF generated within the interior of a cone 422 and projected downwardly toward an individual lying below, is a left handed ISF. As illustrated further in FIG. 21b, distribution assembly 422b is disposed along the central axis of hexagonal projector 422 and comprises a hexagonal star plate 422c the same as or similar to collector plate 418 of FIG. 2g. Star plate 422c is coupled through wires 422d to each of the six outer cones 422a. Cylindrical cone 422e is clamped to star plate 422c using gasket 422f, which is affixed to a main body of projector 422 using a series of three bolt holes. Gasket 422f holds cone 422e down, centered on star plate 422c. In one embodiment, the main body is made of masonite, gasket 422f is birch plywood, and the bolts and nuts are nylon. FIG. 25, discussed further below, presents an alternative distribution assembly 340a, in accordance with a further embodiment of the present invention.

As illustrated in FIG. 21c, hexagonal projector 422 can include a side housing 422g and top plate 422h designed to impart an outer appearance of a simple hexagonal prism shape to projector 422 when assembled.

In one configuration of the present invention illustrated in FIG. 21d, projector 422 includes a carriage assembly 422i, which includes a top plate portion 422j and chassis portion 422k. Chassis portion 422k is configured with a set of eight wheels 422l, which are designed to couple to an aluminum rail provided in a top assembly between the two end structures, as described above. Accordingly, projector 422 can be suspended from a top assembly 424 moved in a horizontal direction above bed 402 to position the projector as desired. Moreover, the axis of hexagonal projector is preferably orthogonal to the axis of tubes 412. Projector 422 is thereby configured to project a statically derived ISF into the region where an individual is located when the individual is lying on bed 402. As discussed further below, cone 422e is configured with a wire coupled to its apex that can be used to conduct dynamically generated ISFs to projector 422.

In another embodiment of the present invention, a ball radiator structure 450 is also provided in destressing apparatus 400, as illustrated in FIG. 22a. Ball radiator device 450 preferably includes a cylinder 452 (e.g., PVC tube) that contains a metallic sphere such as copper/silver alloy or copper (e.g., ⅝ inch diameter copper ball), which is also slidably moveable in a plane parallel to that of tube 412, using a top portion 454 (e.g., a wooden box made from ¼ inch birch plywood) provided with a cylindrical through hole as shown in FIG. 2a. As shown, cylinder 452 is pivotally mounted to top portion 454. Accordingly, ball radiator 450 can be positioned over and adjusted for an individual, for example, focusing the ball radiator 450 on the “third eye” chakra in the region of the forehead, as illustrated in FIG. 22b. A static ISF generated on the outside of the sphere defined by the ball irradiator 450 is a right handed ISF. As discussed further below, ball radiator 450 is configured with a wire 456 (e.g., 16 gauge multi-strand coiled copper speaker wire) that can be used to conduct dynamically generated ISFs to radiator 450. Wire 456 can be connected through a gold-plated copper butt terminal to a wire inside cylinder 452 (e.g., a 10 gauge single strand copper wire) that is brazed (e.g., with 72% silver and 28% copper alloy braze) to the side of the metallic sphere.

FIG. 2k is a schematic perspective view of a system 200 for destressing, in accordance with another embodiment of the present invention. System 200 includes a bed 202 that is affixed at each end to end members 204 and 206. In this embodiment, end members 204 and 206 each comprise a bilayer structure, 204a, 204b, and 206a, 206b, respectively, which preferably comprises a wood-based material.

Sandwiched between each bilayer structure is a star pattern 208, preferably comprising conductive tape arranged to form a Star of David-type geometry, using a metallic tape such as copper tape. Similar to star pattern 408, star pattern 208 comprises apices 210, as also illustrated in FIG. 4. Each star apex is connected to a tube 212 that in turn connects that apex with a corresponding apex in the opposite star pattern. Preferably, each tube 212 comprises an outer metal tube (not separately shown), such as copper and further includes a supporting wooden dowel (not shown) within the metal tube. The tubes 212 are arranged to be substantially orthogonal to the planes of end members 204 and 206. Thus, tubes 212 are mutually arranged in a hexagonal array as viewed along the axis of the tubes. This arrangement serves to define a larger hexagonal prism space 414, as illustrated in FIG. 2b. The base edges 415 of the prism space are defined by connecting adjacent star apices 210. The length of the prism space is equivalent to the length of the tubes 212. The height and relative lateral position of bed 202 is configured such that an individual lying on bed 202 is substantially or wholly within the space defined by prism space 414, as illustrated in FIG. 4.

The metallic tubes 212, together with metallic tape patterns 208 are configured to establish and direct an ISF environment particularly within the region defined by prism space 414, although ISFs can extend into the region outside of prism space 414.

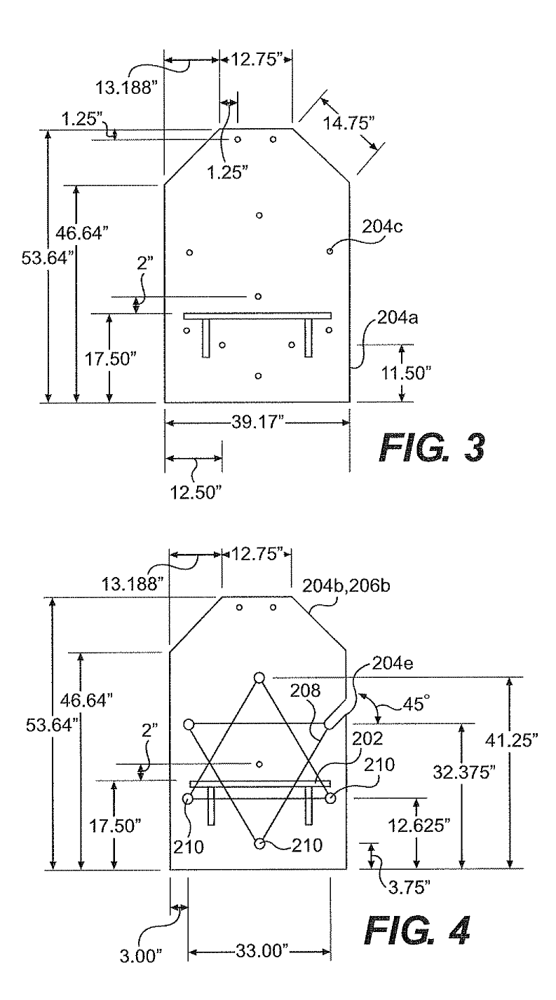

FIG. 3 illustrates details of an end structure outer panel 204a, in accordance with one embodiment of the present invention. As shown, panel 204a comprises a beveled top and flat base.

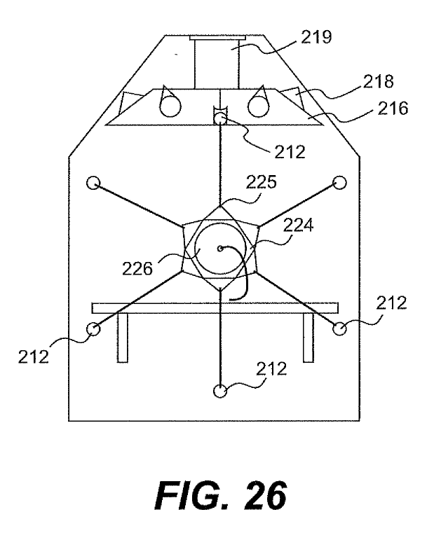

FIG. 7b illustrates back and front views of a foot assembly 222 that is provided over an opposite portion of bed 202 as compared to the location of ball radiator 220 (see FIG. 2k), in accordance with one embodiment of the present invention. In one embodiment of the present invention, foot assembly 222 comprises a ¾″×15″×33″ Luan surface finished plywood foot panel 222a supported by two 4″×15″ braces 222b and a 4″×33″ bottom panel 222c. Copper panels 223 are made of 6″×13.5″ copper sheets, where each panel is offset about 1.5 inches from the center of panel 222a, where each copper panel is designed to rest against the feet of an individual lying on bed 202. In accordance with one embodiment of the present invention, illustrated in FIG. 26, metallic tubes 212 are coupled to the foot assembly 222 using a concentrator 224. Collector 224 preferably is similar to or substantially the same as collector 416 and comprises a metal having a base that is shaped in a hexagonal star pattern whose apices 225 are each coupled to a metallic tube 212 using 16 gauge multi-strand copper wire, which is soldered to copper tape located between panels, such as panels 204a, 204b. The copper wire is fed to the copper tape through holes 204c provided in the panels (see FIG. 3). Concentrator 224 also includes a conical structure 226 whose apex is contacted by a 16 gauge multi-strand wire that feeds through holes provided in inner and outer panels 204b, 204a and leads to a foot assembly, such as foot assembly 222, shown in FIG. 7. A copper wire is fed through assembly 222 from front to back and is soldered to each of the foot panels 223, as shown. Copper foot panels 223 are preferably nailed to plywood foot panel 222a using four copper nails placed at the corners of each panel. Thus, concentrator 224 is configured to concentrate ISF fields from tubes 212 to the apex of cone 226 and to direct the ISF to the region of the foot panels 223.

The metallic tubes 212, together with metallic tape patterns 208 are configured to establish and direct an ISF environment particularly within the region defined by prism space 414.

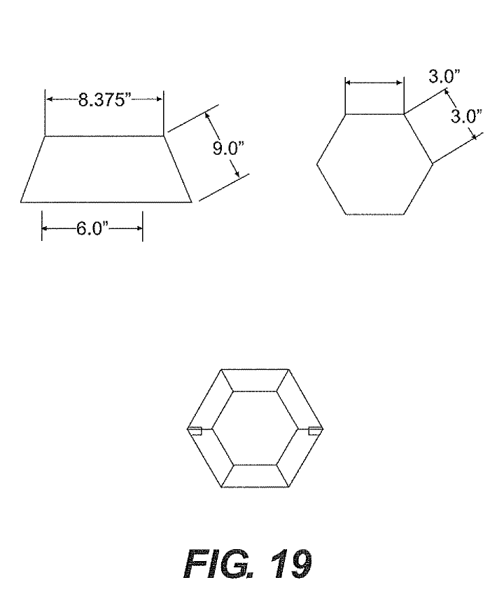

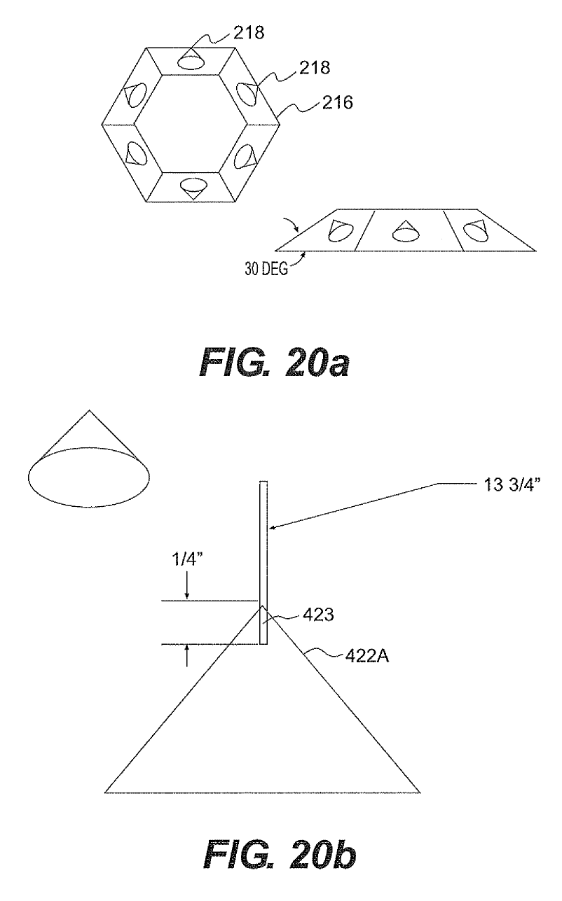

System 200 also includes a hexagonal assembly (projector) 216, which can be used to provide an ISF environment inside structure 202. In one embodiment of the present invention, hexagonal assembly 216 is configured with a series of six cylindrical cones 218, as illustrated further in FIGS. 19 and 20. Preferably, projector 216 is configured similarly to projector 422 such that the cones are mutually arranged so that their cone axes all converge upon a point. Preferably, cones 218 are phi ratio cones in which the ratio of base to height is about 1.618. In one embodiment, illustrated in FIG. 2a, the hexagonal assembly 216 is configured such that the axes of all cones converge at a point (not shown) above bed 202. In embodiments of the present invention, discussed further below, hexagonal assembly 216 is slidable along a tube 212, such that the convergence point can be positioned above a specific region of an individual resting on bed 202, such as the heart chakra region. The arrangement of cones 218 is such that the base of each cone is downwardly disposed (closer to bed 202) with respect to the cone apex.

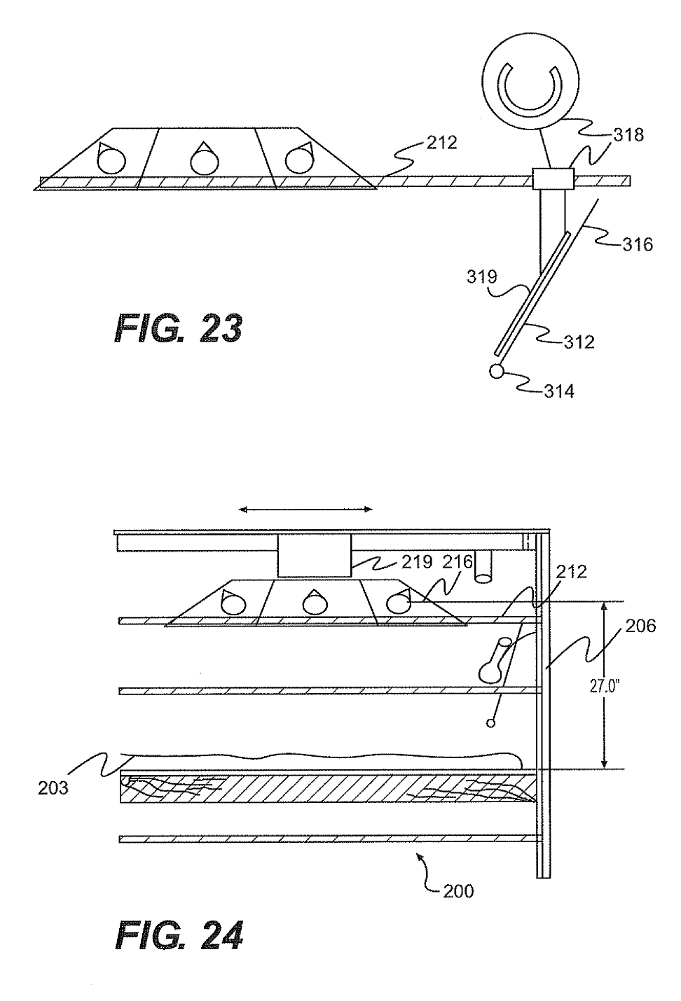

In an embodiment of the present invention, a ball radiator structure 220 is also provided in destressing apparatus 200, as further illustrated in FIG. 23. Ball radiator 220 is preferably a metallic sphere such as copper silver alloy or copper, which is also slidably moveable in a plane parallel to that of tube 212. Accordingly, ball radiator 220 can be positioned over an individual, such as above the “third eye” chakra in the region of the forehead. A static ISF generated on the outside of the sphere defined by the ball irradiator 220 is a right handed ISF.

In accordance with the above-described elements of system 200, static ISFs can be distributed and projected within one or more areas of a spatial region that accommodates an individual on bed 202, such that the ISFs interact with the individual to produce a destressing effect.

FIG. 21 is a schematic illustration that depicts elements of a system 230 for destressing in accordance with additional embodiments of the present invention. System 230 is designed to accommodate an individual in a structure 232 for a period of time to facilitate destressing of the individual. In an embodiment of the present invention, structure 232 includes bed 402, end members 404 and 408, and metallic tubes 412 and star patterns 408, as shown with respect to FIG. 2a. Alternatively, structure 232 includes bed 202, end members 204, 206, tubes 212 and star patterns 208, arranged substantially as shown and described above with respect to FIG. 2k. Structure 230 also preferably includes a hexagonal collector, such as collector 224 or 416 that is coupled to a foot assembly such as assembly 222 or 440. Thus, structure 232 is configured to conduct and distribute ISFs within a region that accommodates an individual in a reclined position. In a preferred embodiment of the present invention, structure 232 is horizontally elongated and bed 202 is horizontal such that the individual is optimally accommodated in a reclined position on bed 202. Alternatively however, as one of ordinary skill in the art would appreciate, the structure could be adapted for other orientations, such as to accommodate an individual who is standing.

System 230 includes an electromagnetic source 234 that can be used as an input for generating ISF inputs. As discussed in detail below, electromagnetic source 234 may include electrical or electromagnetic outputs from a music source, such as a CD, audiotape, and the like. Alternatively, electromagnetic source 234 may include an SCR controller or similar device that can control a light source, such as a lamp.

Electromagnetic or electrical signals from electrical source 234 are conducted to dynamic ISF generator 236. ISF generator 236 is configured to receive the electromagnetic or electrical input from electrical source 234, which can be used as an input to cause ISF production by dynamic ISF generator 236. In a preferred embodiment, both electromagnetic source 234 and dynamic ISF generator 236 are located in a region external to structure 232.

System 230 is also configured such that the electrical or electromagnetic signals received from electromagnetic source 234 are substantially blocked or attenuated from propagating into the immediate environment of an individual in structure 232.

An information spin field generated by ISF generator 236 is conducted along ISF conductor 238 to ISF projector system 240. ISF conductor 238 can comprise, for example, an electrical conductor, such as a metal. Alternatively, ISF conductor 238 can comprise an insulator material, such as an optical fiber. In embodiments of the present invention, system 230 further includes an attenuator (or coupler) 242 that acts to conduct ISF into structure 232, while preventing electromagnetic or electric signals from propagating from ISF generator 236 to ISF projector system 240. In other embodiments, however, an attenuator 242 separate from the ISF generator 236 need not be included. This is because the ISF generator 236 is preferably configured to prevent electromagnetic or electric signals from propagating to projector system 240, as discussed further below. Thus, electromagnetic or electrical signals that are used as inputs to ISF generator 236 or are byproducts of ISF generator 236 during its operation, are substantially blocked from propagating into areas such as areas A, B, and C of structure 232.

As discussed further below, ISF projector system 240 may be configured to distribute dynamically created (and statically created) ISFs from multiple positions toward the vicinity of the individual, or alternatively, may be configured as a relatively localized single source that radiates ISF in the vicinity of the individual during a destressing session. ISF projector system 240 may comprise a plurality of separate ISF projectors (as depicted, for example as 240a and 240b), that comprise similar or different features, and are directed at different regions near an individual, as discussed in detail in the discussion to follow. For example, an individual ISF projector might include a series of identical structures, such as cones that are mutually arranged according to a predetermined geometry within the ISF projector. Alternatively, an ISF projector system might include two or more ISF projectors that differ in structure and materials, and are interconnected with different elements, such as different ISF generators. The term “ISF projector,” as used herein, refers to an object or system that provides or directs an ISF or set of ISFs within a desired region, for example, in a region of a destressing structure than can accommodate an individual. As depicted in FIG. 2c, projector 240 is located within structure 232, but need not be located within such a chamber.

A set of dynamically generated ISFs is provided by projector system 240 in a manner that enhances the destressing of an individual located within structure 232. Accordingly, the individual resting in structure 232 receives the benefit of an ISF environment purposively created from sources that can create harmonious ISFs without any unwanted or deleterious effects associated with the electromagnetic or electric sources associated with generation of ISFs themselves. In preferred embodiments of the present invention discussed further below, projector system 240 comprises a ball radiator and hexagonal assembly (each discussed previously). Projector system 240 thus may comprise components that are configured to project both statically generated and dynamically generated ISFs in the region of structure 232 surrounding an individual.

As described further below, in some embodiments of the present invention, a dynamic ISF generator can be switched from generating right handed ISFs to generating left handed ISFs. Additionally, as noted above, different elements of system 230, such as the ball radiator and hexagonal cone assembly produce either left handed or right handed static ISFs. When dynamic ISF generators are employed, the ISF environment thus established in the environment of the individual in structure 232 results from a combination of dynamically generated ISFs as well as statically generated ISFs, whose intensity and handedness may differ. In embodiments of the present invention in which a source for dynamic ISF generation is employed, the intensity of the dynamically derived ISF tends to be such that the dynamically derived ISF exercises a dominant effect on the ISF environment established in the vicinity of the reclining individual, such as regions A, B, and C.

Referring again to FIG. 21, system 230 further includes monitor 244, which can be used to monitor the response of an individual during a destressing session, which can aid in tuning the ISF input during a destressing session or adjusting ISF inputs for future sessions. This is useful so that the energetic input used to generate ISFs can be tailored to the individual to optimize the destressing effect for that individual.

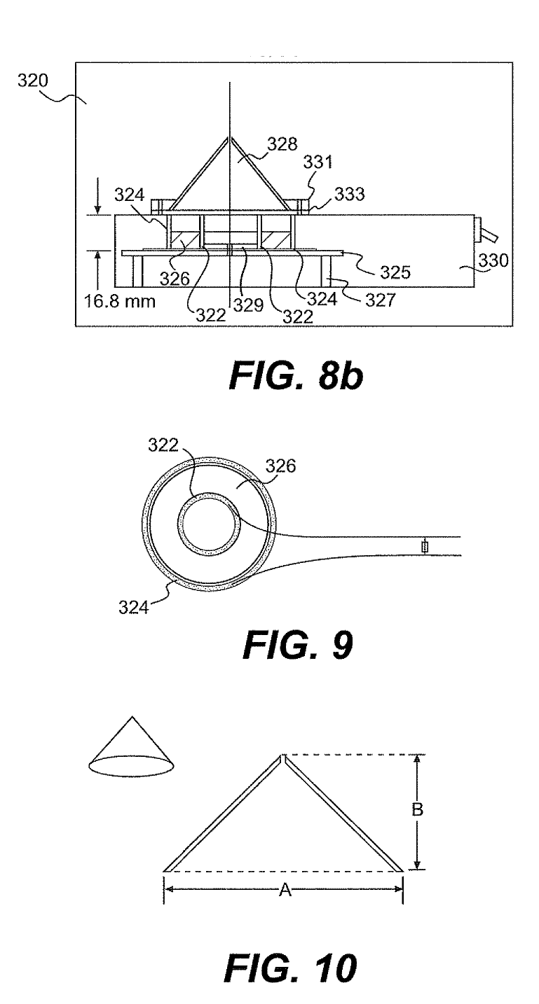

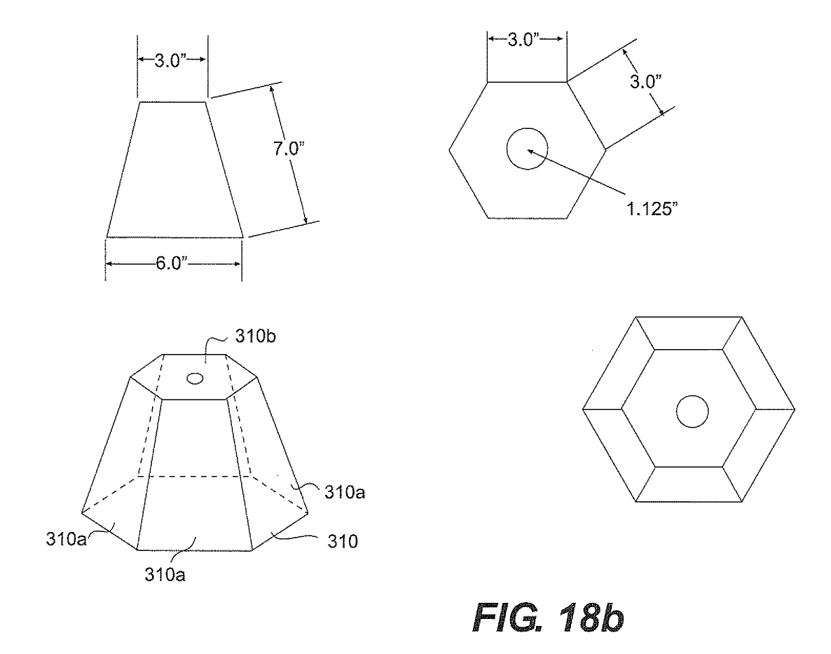

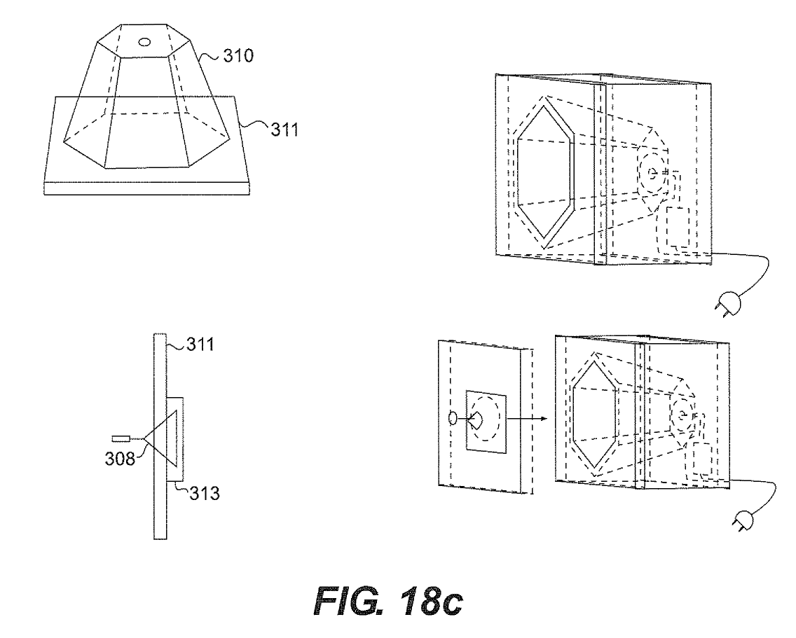

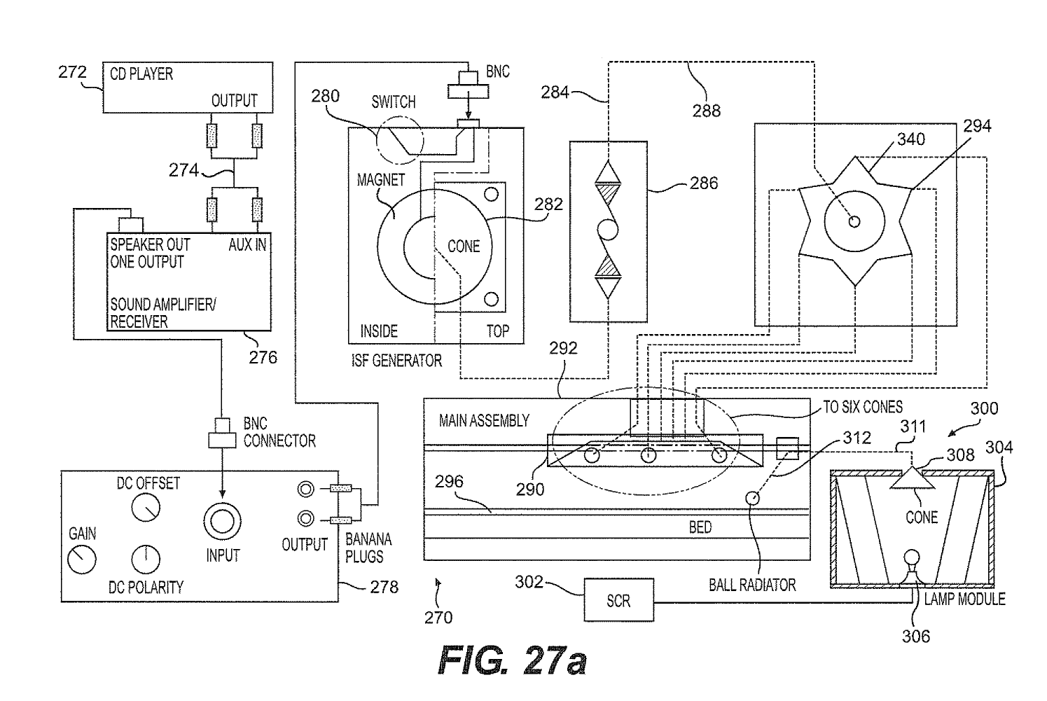

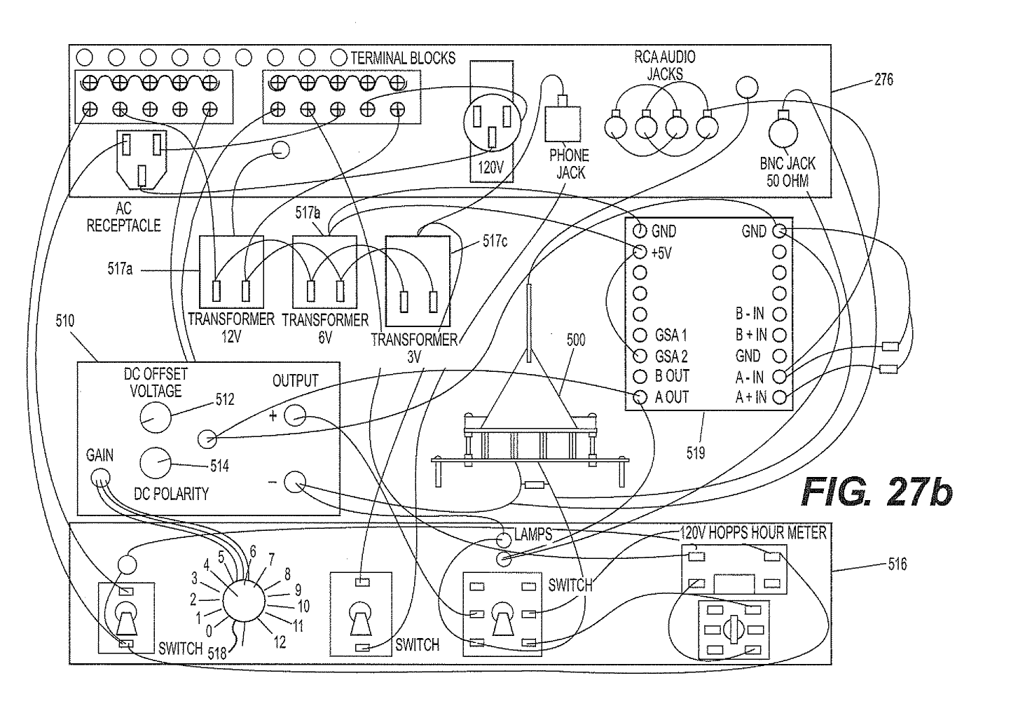

FIG. 2m is a schematic illustration that depicts the interconnection of elements of a system 460 for destressing in accordance with another embodiment of the present invention. Solid lines show flow of electromagnetic currents and dashed lines show ISF flows. System 460 includes main assembly 462 that contains bed 464 and hexagonal projector 466 and ball radiator 468 located above bed 464. CD player 470 is configured to play, for example audio CDs that output an electronic signal to receiver 472, which outputs a signal to high voltage amplifier 474. Amplifier 474 in turn, outputs a signal to dynamic ISF generator 476, examples of which are described further below with respect to FIGS. 8a and 8b. ISF generator 476 is connected to fiber coupler 478 that is designed to block electric and electromagnetic signals from propagating to assembly 462. Fiber coupler 478 is connected to distribution box 480, that may include a distributor such as distribution assembly 422b depicted in FIG. 21b. Distribution assembly is configured to receive ISFs generated by ISF generator 476 and distribute them within hexagonal projector 466. Accordingly, dynamic ISFs produced from inputs derived from CD player 470 are projected within assembly 462 by hexagonal projector 466. In addition, ball radiator 468 is configured to receive ISFs generated by the action of SCR 482 which outputs a signal that controls the intensity of light in lamp module 484. The light generated in lamp module 484 is collected and blocked from leaving the lamp module, as described further with respect to FIGS. 18a-18c. ISFs produced by lamp module 484, on the other hand, are conducted to ball radiator 468 and projected into assembly 462. As described further below with respect to FIG. 27a and 27b, the ISF environment created within an assembly, such as assembly 462 can be used to facilitate destressing.

In one embodiment of the present invention generally depicted in FIG. 1 and more particularly in FIG. 27a herein, destressing is accomplished by using an electromagnetic signal comprising frequency information derived from music as an input to a dynamic ISF generator. This is accomplished in that particular embodiment by using the output signal from a CD player as input to a high voltage amplifier of a type typically employed for powering piezoelectric transducers, with the output of the amplifier feeding as an input to the dynamic ISF generator. Alternatively, either a live music or recorded music source converted to an electromagnetic signal provided by an audiotape player, radio, computer storage device, television, or similar device can be employed. Moreover, other harmonious informational sources, such as the sound of ocean waves and wind, can be employed as inputs to one or more ISF generators providing input to the environment of the present invention in a similar fashion. In the case where music is used as the informational basis for the dynamic ISF input to the device, system, apparatus, and method of the present invention, the music upon which such signal is based can be selected as being of a type harmonious to the individual. Examples of music used in such an embodiment are shown in Table 1 below.

TABLE 1

| Artist | Album Title | Publisher |
|---|---|---|
| 2002 | Wings | Real Music |
| Merlin's Magic | The Heart of Reiki | Inner Worlds Music |
| Merlin's Magic | Angel Symphony of | Inner Worlds Music |
| | Love and Light | |
| Aeoliah | Angel Love | Oreade Music |
| Erin Jacobsen | Feather on the | Serenity Music |
| | Breath of God | |
| Chuck Wild | Liquid Mind IV | Real Music |
| Angie Bemiss | Recovery | James Schaller |
| Steve Halpern | Gift of the Angels | Inner Peace Music |
| Merlin's Magic | Light Reiki Touch | Inner Worlds Music |
| W. A. Mozart | Classical Relaxation | Direct Source |
| | with Ocean Sound | Special Products |
| Gerald Jay Markoe | Celestial Mozart | Astro Music |
| Merlin's Magic | Chakra Meditation | Inner Worlds Music |
| | Music | |
| Merlin's Magic | Healing Harmony | Inner Worlds Music |
| Deuter | Reiki's Hands of Light | New Earth Record |

While not wishing to be bound by any particular theory, it is believed that the above music examples have combinations of tones and patterns which create geometric effects which are particularly harmonious when employed in the present invention. A relationship between music and geometry has been observed by Princeton University Theorist and composer Dmitri Tymoczko, among others, who published some of his findings to that effect in The journal Science in 2006.

As a practical matter in choosing music that has harmonious properties desirable for use in connection with the present invention, it has been observed that certain individuals have an ability to perceive that the music has suitably harmonious properties by listening to the music with headphones apart from any presence of such individuals in the apparatus of the present invention. When they are thus listening and the proper harmonious characteristics are present in the music, such individuals perceive a feeling of vibration or tingling that pervades their whole body that is unique to the types of music that are desirable for use as ISF informational sources for use in connection with the present invention. This represents a method employed to choose music employed in the apparatus of the present invention. All of the CD music albums listed in Table 1 were chosen by this means and exhibit such characteristics. By contrast, most music, even though it may be pleasant to listen to, lacks such a property.

FIG. 27a is a schematic illustration that includes a circuit diagram of components of a system 270 for destressing, in accordance with one embodiment of the present invention. The circuit arrangement illustrated can be implemented in a physical apparatus similar to that depicted in FIG. 1. Solid lines show flow of electromagnetic currents and dotted lines show ISF flows. In accordance with embodiments of the present invention, a source of information, such as a signal from a CD player or from a light source, is conducted to an ISF generator, which can comprise a cone. The ISF generator then locally generates an ISF, which is distributed in the environment of a chamber that can accommodate a person.

In the embodiment depicted in FIG. 27a, an information source comprises a CD player 272. The electrical signal from the music information played by CD player 272 is conducted over conductive wire, such as coaxial leads 274 through a sound amplifier 276 to a high power amplifier device 278, which, in one embodiment, is configured to produce an output voltage not exceeding 120 volts when the gain is at maximum. The output of the high voltage amplifier device 278 thus contains information related to the music contained in the CD.

A switch 280 regulates conduction of an electromagnetic signal from amplifier 278 to ISF generator 282. ISF generator 282 can comprise a conical structure, as described further below with respect to FIG. 8.

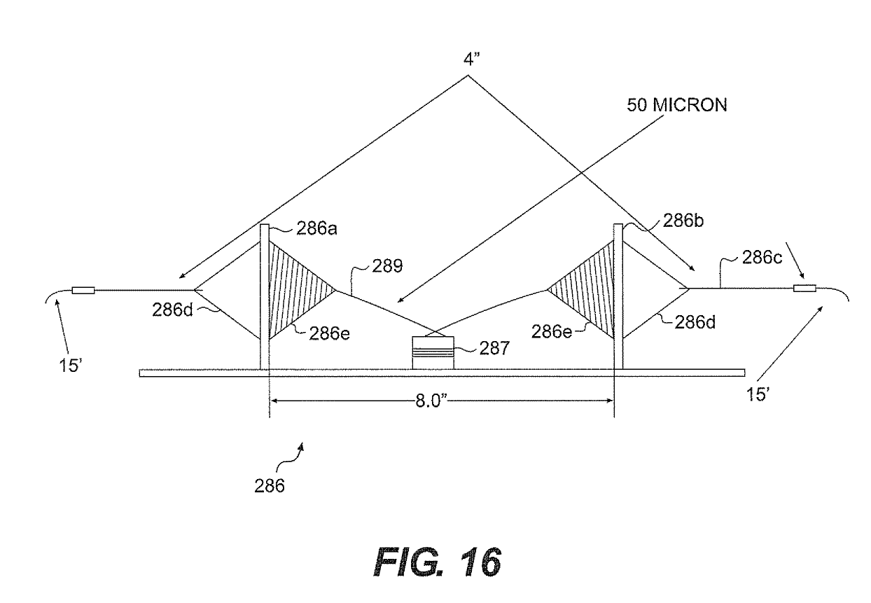

In the embodiment depicted in FIG. 27a, ISF conductor 284 comprises a fiber optical coupler 286 (described further below with respect to FIG. 16) and multi-strand conductive wire 288. ISFs generated from ISF generator 282 are conducted to ISF projector 290, which comprises a hexagonal distribution assembly 340 that may be disposed within main assembly structure 292. A series of six projection points 294 are arranged at tips of cones arranged in a hexagonal array that is disposed directly over bed 296. Accordingly, ISFs can be provided in a region of structure 292 that is designed to accommodate a reclining individual. In this manner, the individual is encompassed in an ISF environment that is provided by projector 290, which receives the ISF from ISF generator 282, which in turn receives an electrical signal based on the music played by CD player 272.

Accordingly, the ISF environment that surrounds an individual in structure 292 is derived at least in part from the information provided by CD player 272. Additionally, as described further below, ISF conductor 284 and fiber coupler 286 are configured to minimize or eliminate electrical and electromagnetic signals derived from the output of CD player 272, such that the individual in structure 292 is subject to an ISF environment substantially stripped of any electromagnetic or electric signals used to help generate the ISF environment. If a metallic material is used to form a conductive wire 288, copper or noble metals are preferably used to form the wire. In some embodiments, instead of a multistrand wire 288, an ISF conductor can comprise an insulator such as an optical fiber.

Another source of harmonious informational input for use in connection with ISF generators of the present invention is light. Such a source can also be provided in the embodiment of the present invention depicted in FIG. 1, and detailed in FIGS. 18a-c, 24, and 27a. FIG. 27a depicts an information source 300 that includes an SCR (silicon controlled rectifier) controller 302 and lamp module 304. SCR 302 acts as an electrical source that is configured to provide a power source to lamp 306 without the AC voltage variation from the line supply. Lamp 306, in turn, acts to generate radiation that is collected at cone 308. The ISFs generated from cone 308 (or combined ISF) are conducted by ISF conductor 310 to ISF projector 312, which is a ball radiator in the embodiment depicted in FIG. 27a. Ball radiator 312, in turn, provides an ISF environment directly in the vicinity of an individual reclining on bed 296. Thus, a light source can be used to generate a harmonious ISF environment in structure 292. In a preferred embodiment of the present invention, ball radiator 312 and hexagonal assembly 290 act in concert with a hexagonal array of metallic tubes (not shown in FIG. 27a, but described above with respect to FIG. 2a) to produce an ISF environment that combines dynamically produced and statically produced ISFs that interact with an individual in structure 292 to produce a destressing effect.

In one embodiment of the present invention, means are provided for determining that the enhanced ISF environment is harmonious to the individual at the time of the individual's presence substantially within the enhanced informational spin field environment. Such a means is provided in the embodiment of the present invention depicted in FIG. 1. It has been observed that an individual who is substantially surrounded by the ISF environment of the present invention exhibits autonomic responses that can be visibly interpreted by an operator. A sensor whose output is directed to a computer programmed to interpret visual data, can also be used to determine whether the environment is harmonious to the individual when present within such ISF environment. Among such autonomic responses are various involuntary eye movements, the most common example of which is blinking of the eyes at a rate much more rapid than normal, which tend to largely cease when the environment is fully harmonious. Such autonomic responses described herein are similar in kind and character to those reported in the published PCT patent application of McNew (WO 2005/058144 and PCT/US2004/042451), observed in the combined sound and light environment described therein. It has been discovered that the phenomenon of such responses are present in the ISF environment of the present invention as well, even in the absence of both light and sound within such environment, and can be can be employed as a means of cueing the adjustment of the ISF environment of the present invention with regard to either its intensity, informational content, or both, to assure the presence of a harmonious ISF environment for the individual within said environment.

In a preferred embodiment of the present invention, a gain knob configured to adjust voltage of the output electromagnetic signal from the high voltage amplifier to the ISF generator may be adjusted downward from its initial setting to eliminate involuntary eye movement on the part of the individual subject if such eye movement is being exhibited either at the commencement of or during a destressing session. If involuntarily eye movement persists, the hexagonal projector may be moved toward the feet of the subject, lowering the overall frequency of the ISF within the destressing apparatus. If involuntary eye movement still persists, this is an indication that the subject has made as much change to their ISF as can be comfortably accommodated at the time of the session, and the session is then terminated by removing the individual from the destressing device.

### Exemplary Implementations of Components of a System for Reducing Stress

In the discussion to follow, details of exemplary components of a system for destressing are provided. Such components are merely exemplary to provide a better understanding of the operation of the invention, and should not be considered as limiting the scope of the present invention in any way.

### ISF Generators

One embodiment of a dynamic ISF generator of the present invention is illustrated in FIG. 8a.

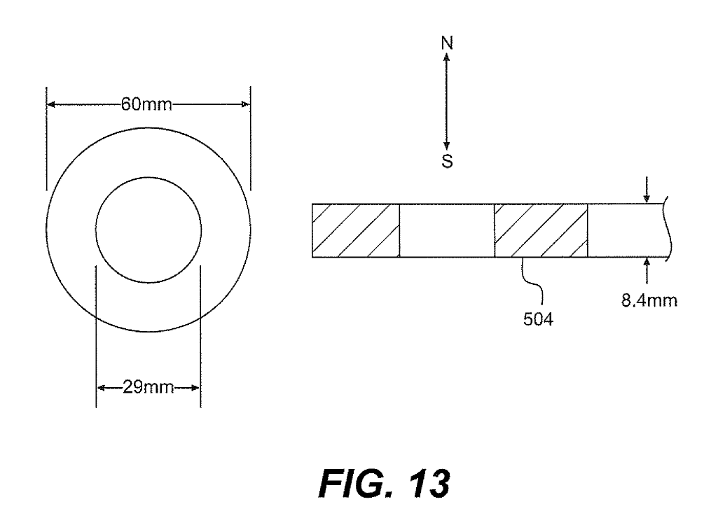

FIG. 8a illustrates a cross-sectional, top, and bottom view of ISF generator 500 that includes cone 502 and an assembly of phosphor bronze and Teflon™ sheets and a ring magnet. A pair of Teflon™/bronze capacitors 506, 508 is concentrically arranged such that a ring magnet 504 is concentrically disposed between the concentric coiled capacitor plates. When a changing voltage is applied to the inner and outer bronze plates 506, 508, the magnetic moments within magnet 504 (the electron spin polarizations of the ferrite ceramic ring magnet) are changing dynamically. This change in spin polarization creates a dynamically changing magnetic field. This dynamically changing magnetic field generates an ISF. This dynamic ISF couples into the static ISF created by the copper cone 502 itself, whose base is affixed to the base 510. The dynamic and static ISF are then broadcast out of cone 502 and follow a copper wire and optical fiber conduction path. In a preferred embodiment of the present invention, as illustrated in FIG. 13, the polarity of the ring magnet 504 is arranged such that the poles are arranged along the axis of the ring and cone, and the north pole is facing up toward cone 502. In one embodiment of the present invention, ring magnet 504 comprises a ferrite ceramic ring type five, with magnetic field strength of about 4000 Gauss. As shown in FIG. 8a, cone 502 is held down by a top square plate and sandwiched between the top square plate and bottom square plate by, for example, nylon bolts through the top and bottom plates.

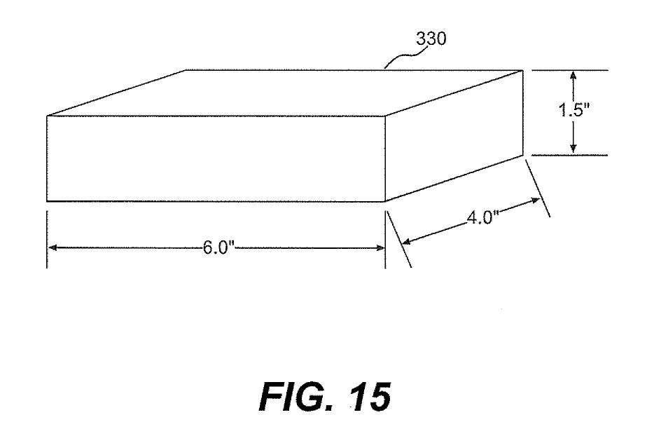

In accordance with another embodiment of the present invention, FIG. 8b illustrates a cross-sectional view of another ISF generator 320 that includes an assembly of bronze and Teflon™ sheets and ring magnets. The embodiment depicted in FIG. 8b differs from that shown in FIG. 8a primarily in that the magnet and capacitors in the former are contained within an enclosure 330. Similar to ISF generator 500, a pair of Teflon™/bronze capacitors 322, 324 is concentrically arranged such that a ring magnet 326 is concentrically disposed between the concentric capacitor plates. When a changing voltage is applied to the inner and outer bronze plates 322, 324, the magnetic moments within magnet 326 (the electron spin polarizations of the ferrite ceramic ring magnet) are changing dynamically. This change in spin polarization creates a dynamically changing magnetic field. This dynamically changing magnetic field generates an ISF. This dynamic ISF couples into the static ISF created by the copper cone 328 itself, whose base is affixed to the outside of a housing 330 that houses the magnet capacitor assembly. An example of housing 330 is shown in FIG. 15.

The dynamic and static ISF are then broadcast out of cone 328 and follow a copper wire and optical fiber conduction path. In a preferred embodiment of the present invention, the polarity of the ring magnet 326 is arranged such that the poles are arranged along the axis of the ring and cone, and the north pole is facing up toward cone 328. In one embodiment of the present invention, ring magnet 326 comprises a ferrite ceramic ring type five, with magnetic strength of about 4000 Gauss.

Preferably, the dynamic ISF field generated by generator 320 or 500 is a right handed field, which can be controlled by choice of polarity of the input signal to generator 320, as illustrated in FIG. 9.

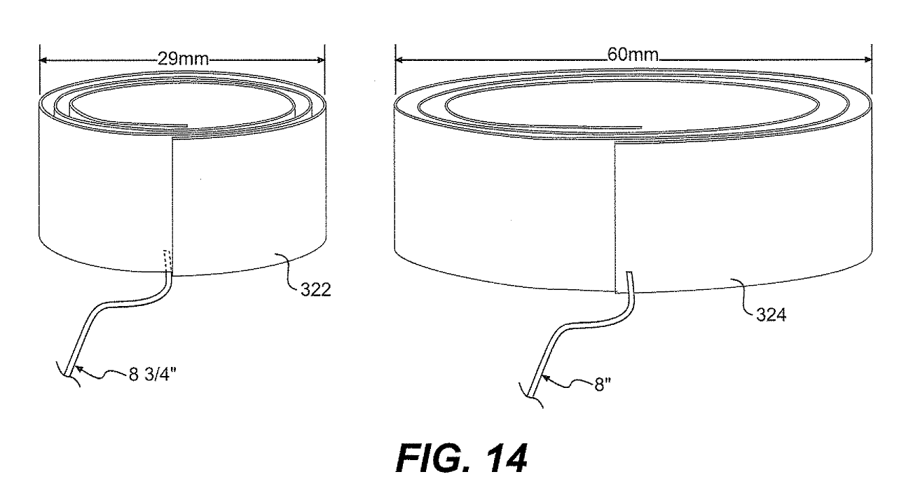

FIG. 14 illustrates details of capacitors 322 (506), 324 (508) according to an embodiment of the present invention. In this embodiment, capacitors 322 and 324 each comprise three concentric layers of Teflon™/bronze formed from a single continuous Teflon™/bronze bilayer. The Teflon™ layer is on the outside. Each layer of the bronze sheet is preferably isolated from the previous and next layer. In one embodiment, the Teflon™ and bronze layers of a single layer of the capacitor are each about 0.005 inches thick. A wire (e.g., 16 gauge 8 inch multi-strand copper speaker wire) is soldered to the inner layer of capacitor 322 and the outside layer of capacitor 324.

Referring again to FIGS. 8a and 8b, the width of capacitors 506 (322) and 508 (324) along the axis of the capacitors and cone is about twice the thickness of the magnet 504 (326).

FIG. 10 illustrates details of a copper cone structure, which can represent, for example, cone 502 of ISF generator 500. An exemplary base diameter is about 60 mm while the height is about 37.1 mm. In one embodiment of the present invention six similarly shaped copper cones are used to form the hexagonal projector described above, as well as cones in a fiber coupler and lamp assembly described below. In the latter cases, an exemplary base diameter is about 101.6 mm and height is about 62.8 mm. Finally, a similar cone structure having diameter of about 63 mm and height of about 39.0 mm is used for a collector assembly connected to a foot assembly described above as well for a distributor assembly connected to the hexagonal projector described previously. Preferably, all such cones are 99.99% oxygen free copper cones.

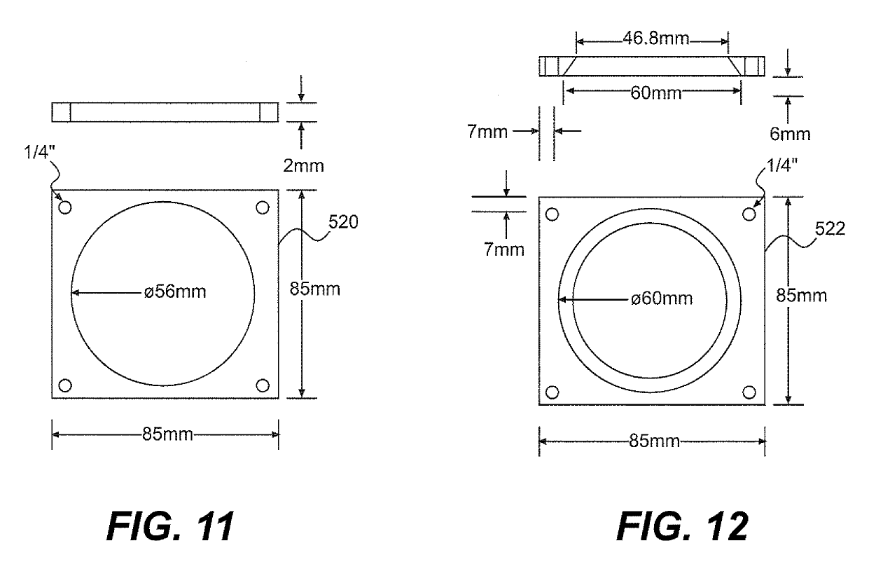

FIGS. 11 and 12 illustrate bottom and top mounts 520 and 522 (e.g., square plates) that are used to fasten cone 502 of FIG. 8a to the underlying magnet/capacitor assembly. Similarly, respective bottom and top mounts 333 and 331 are used to fasten cone 328 to housing 330, as illustrated in FIG. 8b.

Once assembled, ISF generators depicted in FIGS. 8a and 8b are configured to produce ISFs that can be conducted from the apex of the respective cone element to an ISF projector, without conducting substantial electromagnetic radiation therewith. For example, the cones are not electrically coupled through an electrical conductor to the magnet/capacitor assembly. Thus, the ISF generators themselves act as filters preventing electromagnetic radiation from propagating along a path between ISF generator and ISF projector. However, as mentioned above, a decoupler can be provided in the path between an ISF generator and ISF projector to ensure that little or no electromagnetic radiation propagates between the ISF generator and ISF projector.

FIG. 27b illustrates a wiring diagram for a system used to supply a signal derived from a music player to an ISF generator such as shown in the embodiments of FIGS. 8a and 8b. In one configuration of the invention, high voltage amplifier 510, which contains DC offset control 512 and DC polarity control 514, is contained within main enclosure 516 of the high voltage amplifier. Potentiometer 518 is used for gain control but DC polarity and offset is preset and generally not varied by an operator of high voltage amplifier 510, which is used to control inputs to an ISF generator 500. Enclosure 516 is preferably provided with a plurality of vent holes to allow cooling during operation of the high voltage amplifier. FIG. 27b also shows receiver 276 and 12V transformer 517a, 6V transformer 517b, and 3V transformer 517c, dual instrumentation amplifier 519, each coupled to the receiver 276 and console 516 as shown.

In one embodiment of the present invention, the electromagnetic signal inputs coming from the high voltage amplifier are based upon the original musical input from a CD or other music player, as illustrated in FIG. 27a. These signal inputs are attached to the capacitors, and therefore are the source of the changing voltage on the capacitors, which in turn causes the magnetic field to dynamically change in a magnet of an ISF generator, such as magnet 326. Alternatively, high voltage signals in the form of single frequencies, such as from a signal generator, can also be used in the ISF generator of the present invention. The ISF generator itself is useful in various embodiments of the present invention. There is no limit to the frequency of the input, so any signal from a frequency generator from small fractions of a Hertz to Gigahertz frequencies have been shown to work for generating ISFs. Frequencies in the visible light range have also shown to work in the ISF generator of the present invention. Thus, there appears to be no limitation with respect to potential frequency inputs and resulting ISF outputs.

FIGS. 18a-c illustrate further details of lamp module 304 discussed above with respect to FIG. 27a. As previously noted, lamp 306 can receive input from an SCR controller 302 to rectify the alternating current input, and, if desired, control operation of lamp 306. In one embodiment, an SCR controller is utilized as a dimmer to lamp 306, which comprises a 40-watt incandescent light bulb to allow simultaneous variation in both the frequency output range (and therefore informational output) and intensity of the light from the bulb. In the presently preferred embodiment of the present invention, the dimmer switch is employed as a rectifier and is set to its maximum output level, without further adjustments. A hexagonal reflector 310, illustrated in more detail in FIG. 18b, is provided to surround lamp 306 and to generate a static ISF in addition to the dynamic ISF produced by the light from lamp 306 itself. Hexagonal reflector preferably comprises a support structure to which inwardly facing mirrored surfaces are joined on the interior of faces of a hollow prism structure shown. Reflector 310 includes six steeply inclined trapezoid mirrors 310a and one horizontal hexagonal mirror 310b provided with a central hole As illustrated in FIG. 18c, reflector 310 may be joined to or rest on a base 311, which forms a base of module 304. As illustrated, an enclosure 313 is joined to the inwardly-facing side of base 311. Enclosure 313 is configured to allow light to impinge on cone 308 only on the inside surface of cone 308. The combined static and dynamically generated ISF is collected into cone 308, which also restricts the visible light from being emitted from module 304. In one embodiment of the present invention, the ISF created in module 304 is conducted through a single strand copper wire that is brazed into the tip of cone 308 using a 72% silver/28% copper alloy. The ISF is conducted to a copper ball radiator (shown in FIG. 23 and discussed further below). In the embodiment of the present invention depicted in FIG. 1, a multicolored 40-watt light bulb called “The Amazing Rainbow Light,” available from Special F/X Lighting Inc., Hurricane, Utah 84737, is used as the light source, providing particular variations in the ISF output of the light ISF generator as the SCR input to the bulb is adjusted.

### ISF Conductors

ISF conductors useful in the present invention include metals, such as copper, silver, gold, and other noble metals, but preferably should not be (although can be) base metals, such as tin or lead due to their potential distortion of an ISF during conduction. Glass can also be employed as an effective ISF conductor. In one embodiment, 12-gauge multi-strand copper speaker wire can be employed to conduct the ISF from the ISF generators to and from the fiber coupler assembly 286, and from there to the hexagonal projector 290 (see FIG. 27a), as well as from the lamp ISF generator assembly to the ball radiator (element 312 of FIG. 27a). The same type of wire can be used to connect the copper tape from its junctions with the copper tubes with the distribution assembly shown in FIG. 21a, and from the distribution assembly to the copper panels of the footplate assemblies shown in FIGS. 7a and 7b. If solder is employed at any point in the ISF conductance means, copper, silver, gold, or other noble metal alloys are preferred, preferably (although not necessarily) free of base metals such as, for example, lead or tin. In one embodiment of the present invention, machined copper and copper sheet can be variously used for cones in the ISF conductance path, as depicted generally in FIG. 10. Copper tubes and copper tape employed in embodiments of the present invention and described above are also part of an ISF conductance path, although the latter elements are not connected directly to dynamic ISF generators. In embodiments of the present invention, optical fiber, such as, for example, quartz or other glass, or (less desirably) acrylic fiber can be used as ISF conductor.

### Attenuators

In one embodiment of the present invention, a fiber coupler assembly is provided, as shown in more detail in FIGS. 16 and 27a. FIG. 16 illustrates a side view of fiber coupler 286, in accordance with one embodiment of the present invention. The fiber coupler acts to transmit ISFs, which are being conducted to a projector, while blocking the transmission of electrical or electromagnetic signals. The fiber coupler also acts to couple ISFs into and out of the cones along conductor 288. In FIG. 16, a pair of couplers 286a, 286b is separated by takeup spool 287. In one embodiment of the present invention, couplers 286a and 286b comprise double cones having a phi ratio geometries. The effect of fiber coupler 286 is to transition the ISF conductance from copper wire to optical fiber and back to copper wire between an ISF generator (see element 282 of FIG. 27a) and an ISF projector (see the hexagonal projector 290 of FIG. 27a). The purpose of this transition is to provide a positive filter blocking any electromagnetic elements' ability to flow through the ISF circuit. Light cannot be conducted through the copper conductors and electricity and magnetism cannot be conducted through the optical fiber conductors. Only ISFs, therefore, are conducted from the ISF generator to an ISF projector such as the hexagonal projector 290 of FIG. 27a. As illustrated in FIG. 16, a metallic wire leads from an ISF generator (FIG. 27a, element 282) into coupler 286a. Any electrical signal entering into coupler 286a is prevented from flowing further due to the insulating nature of optical fiber 289, which is preferably glass or an insulating polymer. Although optical fiber 289 can transmit electromagnetic radiation such as light, any light entering coupler 286b is prevented from further propagation, because the light does not propagate along metallic wire 286c leading from 286b to a projector. Thus, any ISF generated by an ISF generator and leaving coupler 286 is conducted toward a projector without the presence of an accompanying electromagnetic signal or electrical potential.

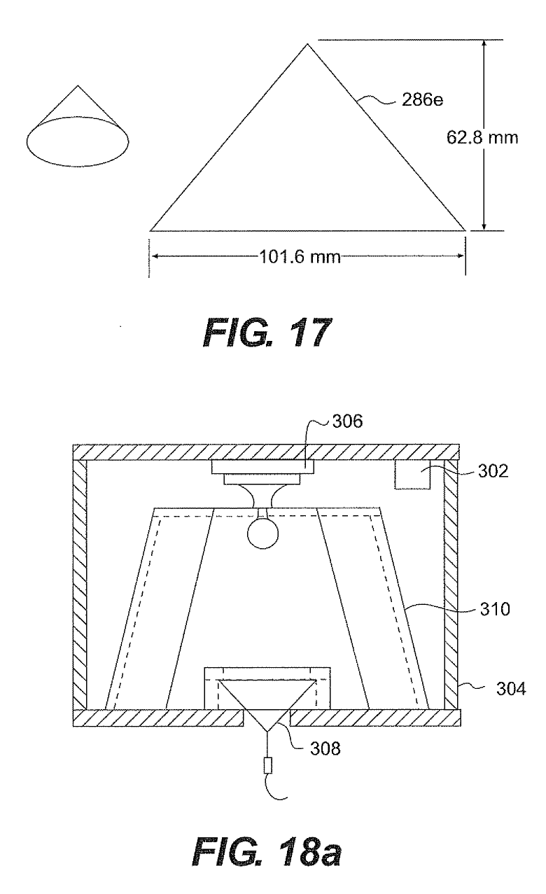

In the embodiment illustrated in FIG. 16, each coupler 286a, 286b comprises a pair of opposed cone structures joined at the base to a common mount. Preferably, the bases of each pair of cones are mutually aligned with each other as viewed down the axis of the cone. Cones 286d are preferably copper cones, while cones 286e can comprise an insulator such as polyester, as shown in FIG. 17. In one embodiment of the present invention, a plywood box (not shown) is placed around coupler 286, with the interior of the box painted flat black.

In one embodiment, cones 286e are polyester and have glass fiber wound 11 turns at a 6 mm pitch as represented in FIG. 16. The direction of the turns is clockwise on the input side, when viewed from the cone's apex and wound counterclockwise, again viewed from the apex of the cone, on the output side. The cones of FIG. 16 can be mounted to plywood such that the copper and fiber wound cones are aligned with each other. The input and output pairs of cones are mounted on a common base for convenience. The fiber is, for example, 50 micron optical fiber. The takeup spool is, for example, a vertical, hollow, plastic spool, 1½ inches in diameter and 1½ inches high, taking up excess of five meter long optical quality glass fiber. The wire 286c can be 10 gauge 4 inch single strand copper wire brazed using 72% silver and 28% copper alloy, with approximately ¼ inch of the wire extending into the cone.

### ISF Projectors

In one embodiment of the present invention, the ISF output from at least one ISF generator is conducted into the ISF environment of the present invention substantially surrounding the individual by means of one or more arrays of copper cones arranged in a hexagonal projector. As discussed above, in one preferred embodiment illustrated in FIG. 21a, an ISF projector 422 (hexagonal assembly) comprises six conical radiators 422a that are employed in a hexagonal array focused upon the vicinity of the center of the heart chakra (approximately the center of the breastbone) of the individual within the ISF environment of the present invention. Cones 422a are preferably objects having hollow geometries, such as, for example, hollow cones having a base to height ratio of phi, approximately 1.618.

FIG. 20b illustrates an example of a copper or phosphor bronze cone 422a, arranged in accordance with one embodiment of the present invention. Cone 422a comprises an approximately 0.005″ thick sheet that is formed into an approximately 61.8×100 mm cone having an approximately 2 mm hole at the apex that accommodates a wire, such as a solid 10 gauge wire, such as copper wire. A length of about 5 mm of copper wire is inserted into the hole and brazed with a low melting point material, such as copper/silver 72%/28% eutectic alloy at an end 423. The joining can be performed using for example a silver/copper alloy described above, which is applied at the end of the wire, after which the wire is soldered to the cone 422a, and the 2 mm hole is sealed. The unbrazed end of copper wire can then be joined to another device, such as a distributor.

The arrangement of cones 422a is such that their bases are facing downwardly when the assembly is affixed in a structure, such as structure 400. As described above, the axes of the cones all preferably converge upon a point below the array that can serve to project dynamically created and statically created ISFs in a region in the vicinity of the heart chakra of an individual lying in structure 400, as illustrated in FIG. 2a.

ISF projector 422 can also include a distribution assembly 422b (340), an embodiment of which is illustrated in FIGS. 21a and 27a. In the embodiment of the present invention depicted in FIG. 27a, distribution assembly 340 is connected to conductor 284 and receives a dynamically generated ISF from generator 282. As further illustrated in FIG. 21b, distribution assembly 422b comprises a metallic star shaped base 422c affixed to a metallic cone, in which the apices of the star point to the points of the hexagonal top plate. Conductor 284 is coupled to the apex of cone 422e. Attached to each point of star shaped base 422c are wires 422d that each lead to an individual cone 422a, depicted in FIG. 21a. Accordingly, the dynamic ISF received from ISF generator 282 is distributed to each cone on assembly 340.

In one embodiment of the present invention depicted in more detail in FIG. 24, a hexagonal projector 216 is configured to slide in a horizontal direction along tube 212, which is an uppermost tube of an array of tubes connecting end members 206 within structure 200. Accordingly, the position of hexagonal assembly 216 can be adjusted according to an individual's size, so that it can be maintained over the heart chakra or other area of individuals of varying height. The centers of bases of cones 218 are located on a common plane that is about 27.0″ above bed 202.

FIG. 23 illustrates details of a ball radiator ISF projector 312, according to one embodiment of the present invention. Ball radiator 312 comprises a copper ball 314 located at the end of a multi-strand wire 316. In one embodiment, the diameter of the copper ball 314 is about ⅝ inch. As discussed previously, ball radiator 312 is connected to an ISF source that employs a light source. Ball radiator 312 is mechanically coupled to hollow tube 318 so that the position of copper ball 314 can move along a horizontal direction when hollow tube 318, which is configured to slide along copper tube 212, is moved. Thus, ball radiator 312 can be adjusted to remain in the same relative position with respect to the head of individuals of varying height.

### Individual Monitoring Equipment

In one embodiment of the present invention, a video camera is provided to furnish observational input of the individual to an operator (via a monitor) or to a computer, for manual or automated employment, respectively, in adjusting the ISF inputs to the environment to achieve harmony and therefore maximize stress alleviation for the individual substantially within the ISF environment. If no visible light is present within the environment, either a passive infrared-sensitive video camera of sufficient resolution or an IR video camera and an IR light positioned, for example, as shown in FIGS. 2a and 2k, may be provided for such purpose. Also located in structure 200 (400) are IR light source 215 (427) and IR camera 217 (425). Light source 215 can provide sufficient illumination inside structure 200 so that IR detector can detect images of objects within structure 200, including details of an individual reclining in structure 200. IR light source 215 is configured to produce radiation of frequency and intensity to cause minimal disturbance to an individual in structure 200. Accordingly, the individual can be observed during exposure to the ambient ISF environment without undue disturbance. In other embodiments of the present invention, other sensors may alternatively be substituted for a video camera as aids in adjusting the ISF inputs to the ISF environment of the present invention to assure that it is harmonious for the individual.

Monitoring of individuals, such as observation of eye movements is helpful in ascertaining an appropriate duration of destressing session. When a dynamically created ISF is provided to an individual, a destressing process may begin to take place over a short period of time, for example ten to twenty minutes. The destressing may be associated with reconfiguring of the individual's own biofield, such that the individual experiences conscious sensations, such as a feeling of relaxation. Autonomic responses such as involuntary eye movement are believed to be an indication that the adjustment taking place in response to the ISF is no longer comfortable. As discussed above, this may be due to an inharmonious ISF environment usually because the intensity of the ISF is too high. However, such autonomic responses observed after a period of time may indicate that the individual is no longer able to accommodate further biofield readjustment comfortably during that session. Thus, a residence time of individuals in the destressing system can be adjusted according to observed indicators, such as involuntary eye movement. The intensity of the ISF projected toward an individual can be lowered by adjusting a high voltage electromagnetic signal input to a dynamic ISF generator.

### Other Hardware

In one embodiment of the present invention, an audio speaker or set of speakers is provided that is coupled to a music source, such as source 272 in FIG. 27. The audio speaker receives an electrical signal from an amplifier and, at the option of the individual subject, can project audible music into the environment of such individual located in system 200. The music corresponds to the same electrical input sent to an ISF generator, such as generator 282, which electrical signal is then blocked from propagating toward the individual along the ISF conduction path. The electrical input into the speaker or set of speakers is transformed into sound by transducers in the speakers. Accordingly, very little, if any, electrical or electromagnetic energy is transmitted from the speakers toward the individual. Preferably, the set of speakers is located outside of the region containing the individual and the ISF projectors.

The present invention offers potential of improved efficiency as compared to means of achieving stress reduction by practices of the prior art. Significantly positive results are observable in a 15 to 30 minute exposure to the informational spin filed environment of the present invention. Individuals experiencing the ISF environment of the present invention typically report feeling a sense of stress reduction, revitalization and wellness. In addition, they often report subsequent healings apparently as a result of being destressed.

While not wishing to be bound by any particular theory, it is believed that consciousness effects facilitated by the environment created within the apparatus of the present invention precipitate the destressing results experienced by individuals spending one or more sessions therein. The following is a non-binding explanation of how and why this is believed to occur.

The human biofield is an ISF whose information content is comprised of ideas or thought forms derived from both the waking (or rational) and subconscious levels of consciousness or awareness. Information inputs to this field from the rational level occur continually as thought and emotion occurs within that level of consciousness, creating content that tends to be transient, except to the extent adopted by the subconscious as part of its evolving self-identity and belief systems. Information inputs to the biofield from the subconscious level tend to be more long term in their tenure in the field, representing fundamental attitudes and convictions adopted by the subconscious concerning the individual's self-identity and worldview. Stress in an individual occurs as a result of: a) negative experiences which are not resolved and are adopted as part of an individual's self-identity as beliefs of having been somehow injured, and b) the exposure of an individual to fears and ideas of limitation about themselves which they do not reject but to which they have come to believe themselves to be subject, and accept as part of their self-identity. Such adopted negative aspects of identity (stress) are then reflected on an extended basis in the ISF that is the biofield of the individual as disharmonious information content.

The biofield is the medium by which the consciousness of an individual communicates with and directs the cellular and biophysical activity that creates and maintains the individual's physical presence. When disharmonious information content (stress) appears in the biofield on other than a transient basis, it becomes part of the instructions that direct the creation and maintenance of the individual's physical body, and becomes manifested as physical disharmony in the form of disease and dysfunction. Disease and dysfunction can be seen, therefore, as the efforts of one level of consciousness (the subconscious) trying (by means of physically manifested disharmony) to get the attention of another level of consciousness (the waking, or rational, level), to get it to recognize and resolve (heal, or discharge) a corruption of the harmony of the individual's self-identity.

When one enters the very powerful and harmonious ISF existing within the environment of the present invention, the subconscious of the individual becomes instantly aware of that field, as well as its greater degree of harmony as compared with the field that the individual has himself or herself created. This awareness causes a response in the individual in which the level of their subconscious then connects with the level of their superconscious (the highest level of their awareness, which is omniscient), in order to try to understand what is occurring. During that connection, the subconscious becomes aware of the specific elements of disharmony that it has adopted into the biofield which it has created, and begins to eliminate those disharmonies issue by issue, resulting in the de-stressing of the individual. As stress disappears from the field of the individual over a series of destressing sessions, they naturally progressively resume a more harmonious physical and mental state, as their innate self-healing mechanisms operate unimpeded by accumulated stress.

Various therapies involving the direct use of light, color and sound on individuals have found a need to vary the frequency inputs specifically to needs of the individual at the particular time of treatment in order to be effective or beneficial. While indeed it is possible to input frequencies of information tailored to address the current needs of a specific individual using the present invention, it is believed to be unnecessary. In the preferred embodiment of the present invention, only the intensity of the field is typically being adjusted, so as not to overwhelm the individual and so as to be of sufficient intensity to facilitate the consciousness connections above described. The music and light frequency inputs chosen are universal. (For example, any of the music sources listed in Table 1 can accomplish the facilitating environment of the present invention.) A key aspect of this modality of the present invention is that the need to choose or structure specific individualized informational inputs is absent: the informational changes necessary to destress the individual are coming directly from within themselves from their highest level of consciousness, which is omniscient and therefore incapable of harming them by introducing inappropriate inputs. Essentially, the present invention creates a facilitating environment where the individual is progressively “remembering” their perfect state devoid of the accumulation of disharmonious experiences and limitations, progressively jettisoning aspects of self-image that do not fit harmoniously. This is often one of the goals of meditators, namely to silence their lower levels of awareness and connect with their highest levels of awareness to become more aware of harmony. Indeed, it has been observed by individuals experienced in regular meditation that being in the environment of the present invention is “like meditating with the static removed,” and that following even a single session in the harmonious ISF environment of the present invention that achieving meditative states thereafter seems easier than before.

There are numerous modalities for healing that operate by introducing various types of informational intervention and/or programming of the individual. These inevitably require receptivity and willingness to accept such informational changes on the part of the recipient. Some of these modalities operate at the level of the subconscious and some directly at the level of the human biofield, to eliminate or otherwise compensate for informational influences that have their origins in stress. These include hypnotherapy, acupuncture, qigong, pranic healing, Reiki, and homeopathy. All of these rely to some degree on the skill of the practitioner in either diagnosis, treatment or both, and in certain circumstances may present various potentials for either ineffectiveness or perceived harm to the individual if the informational inputs are inappropriate to the need.

A preferred embodiment of the present invention comprises a method for achieving destressing of an individual without any necessity for diagnosis or treatment by a practitioner. Such method comprises placing an individual in an environment into which both statically derived and dynamically derived ISF elements are present, from which the electromagnetic components have been removed from at least one such dynamic ISF element.

One example of such a preferred embodiment can be accomplished in the apparatus described above. An individual lies on the mattress of the bed for typically a 20 minute session, during which time a dynamic ISF derived from a musical source with appropriate harmonious characteristics (such as, for example, one of those illustrated in Table 1) is provided in addition to one or more static ISFs. Such dynamic source is adjusted downward in intensity if necessary to assure that no involuntary eye movement is being exhibited by the individual within the apparatus. The subject will often afterward report perceptions of tingling or other sensations in the body, and perhaps colors and/or visions observed mentally. Upon emerging from the session feelings of renewal and revitalization are commonly reported. Subsequent observations of later healings are often reported as well, believed to be the result of destressing. Occasionally increased abilities are later reported to be manifesting, such as the ability to perceive ISFs visually as colors, spontaneous receiving of correct but previously unknown information, premonitions, and increased awareness.

A characteristic of the ISF that is the human biofield is that it has both right handed and left handed elements, and circulates within and surrounding an individual's physical body. Disharmonious information contained in the biofield manifests as blockages in the normal flow of the ISF. The science of acupuncture is directed at the intervention at acupoints to attempt to unblock such flow blockages. Disharmonious information contained in the biofield also manifests as imbalance in the parasympathetic and sympathetic elements of the autonomic nervous system. In connection with the present invention, it is postulated that the progressive abandonment of negative elements of self-identity by the subconscious as a result of connecting with the superconscious in the ISF environment of the present invention appears to result in the removal of blockages to the ISF flow of the individual's biofield. The ISF of the present invention is itself observed (by those who can either feel them or perceive them visually) to circulate more or less along the longitudinal axis of the hexagonal prism space, radially out at the bulkhead at one end, and back along the copper tubes to the other bulkhead, then radially inward and then back through the middle of the prism space along its longitudinal axis. This circulation appears to occur despite no means being deliberately introduced to cause such circulating flow. The ISF flow has been observed to vary in direction (from head to feet of the individual, or vice versa) with different individuals, but has been perceived to be harmonious.

The destressing device of the present invention is preferably located in a quiet setting. A typical procedure for conducting a destressing session in the apparatus of the present invention is as follows:

The operator turns on power to the main electronic console, including the lamp ISF generator assembly, CD player, infrared camera, video monitor, and infrared light (if needed—ambient room lighting may be sufficient to not require the IR light for the camera). A CD music recording such as one of the albums described in FIG. 1 is placed in the CD player. The client individual removes shoes, metal, jewelry and eyeglasses to the extent feasible. The removable copper tube (entrance tube) on the side of the destressing device is removed by the operator, and the hexagonal projector is slid to the far left extreme of its travel within the hexagonal prism space of the destressing device. The ball radiator assembly is slid to the extreme right of its travel within the space. The client individual then enters the prism space, lying on their back with their head to the right, their feet to the left, and their arms at their sides with their body substantially aligned in the direction of the axis of the prism space. One or more pillows and/or a blanket may be provided for the comfort of the individual. The ball radiator is then slid to the left and positioned so that the copper ball is above the vicinity of the “third eye” chakra (the middle of the forehead region an inch or so above the eyebrows) of the individual. The hexagonal projector is slid to the right and positioned such that its center is above the vicinity of the heart chakra (approximately the middle of the breastbone) of the individual. The two foot paddles are strapped to the bottoms of the individual's feet using the Nylon® hook and loop straps attached to them. The entrance tube is then replaced into the destressing device.

The individual is offered the choice of hearing the music from which the ISF will be derived or not. If the individual elects to hear it, a switch is enabled which will route the electromagnetic audio signal from the CD player to small speakers located in the upper right quadrant of the destressing device, above the prism space, in addition to the signal still being conducted to the ISF generator. (An adjustable volume control for the speakers is located on the right bulkhead of the destressing unit at the edge of the prism space within reach of the individual.) The operator then pushes the “play” button of the CD player, activating its electromagnetic audio signal output. The “gain” knob which controls the ISF strength of the output of the hexagonal projector is set by the operator at a value of “3” as marked by its dial. The switch at the panel of the electronic console which activates the high voltage amplifier (main “field switch”) is then turned on by the operator, empowering the ISF generator and its output which feeds the fiber coupler and hexagonal projector.

The operator then looks at the video monitor screen to determine whether the ISF field strength within the destressing device is too strong for the individual within, an affirmative indication being demonstrated by involuntary eye movement of the individual, such as rapid blinking of the eyes. If such eye movement is observed, the operator promptly reduces the gain until the individual's involuntary eye movement response is eliminated. If the involuntary eye movement persists regardless of the gain setting being at its minimum, the session is promptly ended by flipping off the field switch, detaching the foot paddles, sliding the hexagonal projector and ball radiator back to their extreme positions, removing the entrance tube, and assisting the individual's egress from the prism space, and then replacing the entrance tube in the destressing unit. (While the entrance tube is removed from its normal registry with the geometry of the prism space, the ISF within the prism space is less coherent. A property of ISFs is that they increasingly condition space to their informational properties as a function of time; therefore the entrance tube is stored in its regular geometric position in order so as not to condition the prism space somewhat incoherently.) The typical explanation for a prompt termination to the destressing session would be that the individual is still processing physical change fallout from a recent improvement to their biofield, and therefore is subconsciously signaling the need for more time to elapse before attempting more improvement to their field so as to not overwhelm their body with the activity of physical change.

Normal time scheduled between destressing sessions would be at least a week; however, critically ill individuals tend to process change faster and are often scheduled at four day intervals. Assuming no involuntary eye movement is observed and therefore that the session is not terminated immediately, the operator then promptly mentally asks for the protection of the individual from any outside negative mental influence, mentally sends unconditional love to the individual, and mentally expresses gratitude for what is occurring in the session. The individual will typically remain within the destressing unit for a total of twenty minutes in a single session, with the operator checking the monitor for involuntary eye movement every five minutes or so to determine whether the session should be terminated sooner than twenty minutes, in which case at the end of the session the termination procedure is as previously described. The operator asks for any perceptions of the individual during the session (sensations, experiences, observations). If several sessions occur over a few weeks with no perceptions reported by the individual as occurring during the sessions and no subsequent benefits are being noticed in wellbeing or capabilities, the gain setting will be progressively increased by the operator from session to session in 0.5 increments until effects are beginning to be perceived by the individual. Following a session, the operator advises the individual to drink plenty of water for at least the four days following the session in order to allow detoxing and bodily repair processes that tend to follow as a result of destressing to operate unimpeded by lack of hydration.

### Parts Specifications and Assembly Instructions for an Exemplary Destressing System

The discussion to follow makes reference to tables and figures that provide descriptions of exemplary components (e.g., electronic parts), materials, and assembly details associated with manufacturing an embodiment of the present invention. The discussion is presented within the context of the embodiment of FIG. 2k, and the referenced “main assembly” refers to the system generally depicted in FIG. 2k. The ISF generator described below corresponds to the embodiment depicted in FIG. 8b, while the ball radiator corresponds to the embodiment depicted in FIG. 23. Notably, many of the steps listed below for construction of the main assembly depicted in FIG. 2k can be employed for construction of the system depicted in FIG. 2a. Similarly, the ISF generator depicted in FIG. 8a can be constructed according to many of the steps listed below, with the understanding that the latter embodiment does not employ an aluminum housing to contain the magnet/capacitor assembly. In addition, apparatus that include combinations of the components described above are within the scope of this invention. For example, a main assembly constructed according to the steps below could incorporate an ISF generator built in accordance with the embodiment disclosed in FIG. 8a and a ball radiator disclosed in FIG. 22a. Thus, notwithstanding the discussion below with respect to the embodiment of FIG. 2k, one of ordinary skill in the art would appreciate that similar assembly methods could be applied to other embodiments.

1. CD Player:

TABLE 2
CD Player

| Manufacturer: | Philips |
|---|---|
| Model No.: | DVP642/37 |
| Product in Inches (L × W × H): | 9.3 × 17.1 × 1.7 |

4× video up-sampling for improved image quality
192 kHz/24 bit audio DAC

| | |
|---|---|
| Movies: | DVD, DVD + R/RW, DVD-R/RW, |
| | VCD, SVCD, MPEG-4, and DivX |
| | 3.11/4.x/5.x |
| Music: | CD, MP3-CD, CD-R and CD-RW |

2. Sound Amplifier (stereo receiver):

TABLE 3
Sound Amplifier

| | |
|---|---|
| Manufacturer | Yamaha |
| Model No.: | RX 496 |
| Amplification | 75 W per channel in 8 ohms |
| Audio inputs | 6 |
| Audio outputs | 2 |
| (Any stereo receiver with comparable | |
| specifications is acceptable) | |

3. High Voltage Amplifier:

TABLE 4
High Voltage Amplifier

| | |
|---|---|
| Manufacturer: | Piezo System, Inc |
| Model No.: | EPA - 104 |
| Maximum Voltage: | ±200 volts peak |
| Maximum Current: | ±200 mA peak |
| Output Power: | 40 watts peak |
| Frequency Range: | DC to 250 KHz |
| Bandwidth: | Into 1K ohm resistive; 3 db roll-off, |
| | 400 KHz; load: Flat, DC to 300 KHz; |
| | Into capacitive load |
| Voltage Gain: | Variable gain, adjustable from 0 to 20× |
| Phase Shift: | −.083° per KHz, typical Slew Rate |
| | (no load) 380 volts/μsecond |
| Maximum Input Voltage: | ±10 volts peak |
| Maximum DC Component: | ±10 volts DC |
| Input Coupling: | Direct DC coupling only |
| Input Impedance: | 10K ohm |
| Output Coupling: | DC coupling |
| Variable DC Offset: | Normally zero volts. Adjustable |
| | to ±200 volts peak |
| Load Impedance: | Capable of driving any load within the |
| | voltage and current limitations of the |
| | amplifier |
| Output Noise | 2 mvᵣₘₛ with output shorted |
| (300 KHz bandwidth): | |
| AC Power Source: | User settable, 100-130 VAC, 50/60 Hz; |
| | or 200-250 VAC, 50/60 Hz |
| Circuit Protection: | Overload, short circuit and thermal |
| | protection. |
| MECHANICAL | |
| Front Panel Controls: | Gain adjust; DC Polarity selector (+, |
| | 0, −); DC offset adjust |
| Rear Panel Controls: | On/off switch; Line voltage selector |
| Terminals: | BNC for Input (ground referenced); safety |
| | shrouded banana jacks for high voltage |
| | output terminals (ground referenced) |
| Weight: | 6.4 kg (14 lbs) |
| Dimensions: | 12″ (305 mm) long × 12″ (305 mm) |
| | deep × 5″ (127 mm) high |

4. Copper tubing: Schedule 40 copper pipe such as used in plumbing.

5. Copper tape: 3/16″ such as used in stain glass.

6. SCR dimmer: Such as available at any hardware store.

7. Lamp socket: Such as available at any hardware store.

8. Copper sheets: Annealed copper sheets 0.005″ thick.

9. Bronze sheets: Phosphor bronze sheets 0.005″ thick.

10. Ball Radiator: Metal ball. 99.9% copper, solid, 0.631″ diameter.

11. IR Camera:

TABLE 5
IR Camera

| | |
|---|---|
| Manufacturer | MaxMax |
| Model | B&W Bullet |
| Infrared Capable | None/715 nm/780 nm/830 nm/850 nm/ |
| | 1000 nm/XDP Optional |
| Image Sensor | ⅓ CCD 290,000 CCIR Pixels |
| Video Format | B/W EIA or CCIR |
| Operating Voltage | DC 9 V to 12 V |
| Power Consumption | 104 mA |
| Gamma Consumption | 0.45 |
| S/N Ratio | >48 db |
| Sensitivity | 0.1 Lux |
| Resolution | >380 TV Line Horizontal |
| Video Out | 75 ohm, 1 Vp-p Composite |
| Operating Temperature | −10 C. to +50 C. |
| Focal Length | 3.6 mm +− 5% |
| F. Number | 2.0 w/o IR lens |
| Field Of View Angle | Diagonal 92 degrees |
| Weight | 50 Grams |
| Dimensions | 20.7 mm (diam) × 59 mm (long) |

IR Camera power supply:

TABLE 6
IR Camera Power Supply

| | |
|---|---|
| Input Voltage: | 100 to 240 Volts AC |
| Input Frequency: | 50 to 60 Hertz |
| Output Voltage: | 12 VDC |
| Output Power: | 200 mA |

13. IR light source:

TABLE 7
IR Light Source

| Model | Type | Wavelength | Beam Angle | Output Power |
|---|---|---|---|---|
| 5LED880 | Infrared | 880 nm | 18 | 2,500 mW/sr * |

14. TV monitor for IR camera:

TABLE 8
TV Monitor

| | |
|---|---|
| Manufacturer | DuraBrand |
| Model No.: | DWT1304 |

15. Type of Optical Fiber: UV/VIS High OH content fused silica core and cladding. These are a stepped index, multimode fiber with a core diameter of 250 um. Has a polymer buffer on for protection. Fiber ends are not polished. Numerical Aperture: 0.22+/−0.02.

### Exemplary Methods for Building the Assemblies

The discussion below provides exemplary methods for assembling components of a system for destressing in accordance with embodiments of the present invention. To aid in understanding, reference is made to the Figures.

1. Magnet and Cone ISF Generator

To construct an ISF generator, reference is made to FIGS. 8b, 9, 10, 11, 12, 13, 14, and 15.

In one embodiment of the present invention, the following exemplary steps are performed:
1. As illustrated in FIG. 14 and FIG. 8b, cut the Bronze and Teflon™ sheets so that they are 2 times the width of the magnet and can be wound 3 times around the magnet. One set is for the outside of the magnet, the other for the inside. Then, layer the bronze and Teflon™ sheets such that the Teflon™ is layer between the bronze and also insulates the bronze from the magnet.
2. FIG. 9 illustrates connecting of wires to the outer capacitor, which is done by cutting back the Teflon™ sheet on the outside of this capacitor and exposing a small area of bronze sheet. To connect to the inner capacitor, the same technique applies, but in addition a small v shaped cut needs to be made on the outer capacitor since, as seen in the ISF assembly drawing, there is no room between the top of the capacitors and the ISF housing (part 12 in FIG. 15). A one MegaOhm resistor is placed across the inputs. An input voltage of up to 150 V can be supplied, where a positive bias produces a right handed ISF and negative bias produces a left handed ISF.
3. A bulk head BNC connector is mounted to the side wall of the aluminum housing (FIG. 15). The aluminum housing can be sheet metal or purchased from an electronic supply catalog. A SPST toggle switch is mounted next to the BNC connector.
4. A 1 Mega Ohm resistor is soldered across the inputs to the ISF generator, typically in between input connector and the wire soldered to the inner and outer capacitors.
5. The outside diameter of the ring magnet should match the diameter of the copper cone above, as illustrated in FIG. 8b. In FIG. 13, exemplary magnet dimensions include a 8.4 mm thickness, a 60 mm inner diameter design to couple to a 60 mm cone and a 29 mm inner diameter.
6. A Teflon™ sheet is inserted between the top of the capacitors and the ISF housing as well as between the bottom of the capacitors and the aluminum plate underneath.
7. As illustrated in FIGS. 8b, 11, and 12 a copper cone 328 is mounted to the top of the ISF housing with the top and bottom Teflon™ mounts, 331 and 333, respectively. In one embodiment, the cone is about 37 mm×60 mm. Teflon™ screws are used to attach mounts 331, 333 to the ISF housing (see FIG. 8). Bottom mount 333 is each a 2 mm thick 84 mm×84 mm square gasket having a 56 mm diameter circular hole in the center and four 1.6 mm diameter through holes for fasteners spaced 70 mm apart. Top mount 331 has similar dimensions as bottom mount 333, except that the gasket thickness is 6 mm and the circular hole is beveled at a 51 degree angle, such that the diameter decreases from 60 mm at the bottom of gasket 331 to 48 mm at the top of gasket 331.
8. As illustrated in FIG. 10, copper cone 328 can comprise a 1-2 mm thick sheet of 99.99% oxygen free copper formed into a cone whose base diameter is 60 mm, and having a tapped hole configured to accommodate a 1.5 mm or 1/16″ thread screw.
9. Either a copper or bronze screw, or solder, can be used to attach a wire to the top of the copper ISF cone 328.
10. A switch can be inserted on either the + or − input lines so that the ISF can be switched off independent of the other equipment (see FIG. 8).
11. An aluminum base 325 is used to mount the magnet 326. The aluminum base 325 is supported by four Teflon™ standoffs located in the corners. A fiber or Teflon™ washer is used to center the magnet 329. The distance between the aluminum base and top of the housing 330 is twice the thickness of magnet 326.

2. Fiber Coupler

To construct a fiber coupler (also termed coupler), reference is made to FIGS. 16, 17, 20.

In one embodiment of the present invention, the following exemplary steps are performed:
1. Provide an appropriate length of fiber: A 10M long strand of fiber is preferably used.
2. After using the specification in FIG. 17 to create the insulator cones, wind 9 turns in an 8 mm pitch spiral, starting at 0.188″ from the base of the original cone. This offset from the base of the cone is due to the fiber board which is glued to the base of the cone for mounting and strength. Insulator cone 291 comprises a 62.8 mm×101.6 mm cone as shown.
3. Direction of windings: In Assembly 06 drawing (FIG. 16), the input is on the left and the output is on the right. The directions of the windings, when looking down on the apex of the input fiber cone is clockwise and it is counter clockwise on the output cone.
4. Two 61.8×100 mm copper cones of 0.005″ thick copper are built. As illustrated in FIG. 16, the two copper cones are each mounted so that the axis of the two input and the two output cones are aligned. The distance between the base of the input copper cone and the input fiber cone is 3/16″. There is no requirement for the distance between the pair of input and the output cones 286a, 286b. In this drawing they are set 8″ apart for convenience and the extra fiber is wound around a small spool 287 that is disposed between cone pairs 286a, 286b. The takeup spool 287 is a vertical hollow insulator tube having a 1.5″ diameter and height, and having four turns of fiber in the example shown in FIG. 16.
5. The cones are mounted on a polymer foam board, such as a 5″×5″ board.

3. Distribution Box

To construct a distribution box, reference is made to FIG. 25.

In one embodiment of the present invention, the following exemplary steps are performed:
1. Using an annealed copper sheet of about 0.005″ thickness cut out a hexagon pattern having an inner hexagon portion of about 1.84″ distance between opposed sides, with triangular tips extending 0.66″ outwardly from each hexagonal side, as shown in Part 16-01 of FIG. 25. The pyramid shapes that extend from the periphery are not separate but are integral to the whole base plate 342.
2. Cut a piece out of the annealed copper sheet so that a cone 344 having the dimensions shown in Part 16-02 of FIG. 25 can be made. Use solder along the outside seam to form the cone, which has dimensions of about 1″ in height and 1.62″ in diameter.
3. Mount the cone 344 in the center of the base 342, as shown in the bottom left of FIG. 25, and use solder to tack down the edges of cone 344 in 6 places.
4. Soldered an input wire to the tip of the cone 344 in the manner shown in Part 06 (FIG. 20).
5. Solder 6 output wires to the tips of the hexagon pattern.
6. Mount the distribution assembly 340a in a suitable non-metal housing.

4. Hexagonal Projector

To construct a hexagonal projector, reference is made to FIGS. 19, 20, and 21a, respectively.

In one embodiment of the present invention, the following exemplary steps are performed:
1. Cut part 15-02 to 07 from polymer foam board (see FIG. 19).
2. Cut part 15-01 from the same material (see FIG. 19).
3. Assemble these pieces as shown in FIG. 19. Hot glue or any other bonding material can be used to affix parts 15-01 to 15-07 together. Once assembled, the face of every piece forms a 30 degree angle with respect to the bottom plane, as shown in FIG. 20a.
4. Once parts 15-01 to 15-07 are assembled, two slots are cut on opposite tips of the hexagon assembly, as shown in the bottom left of FIG. 19.
5. Assemble 6 copper cones 218 (the term “copper cones 218” also is meant to include phosphor bronze cones, unless otherwise indicated) in the manner shown in the drawing for Part 06 (FIG. 20a). Use solder on the outside seam of the cones.
6. Mount the wires in the manner shown in FIG. 20.
7. Mount the 6 cones 218 in the center of the six faces of the hexagon structure 216.

5. Lamp Assembly

To construct a lamp assembly, reference is made to FIGS. 18b.

In one embodiment of the present invention, the following exemplary steps are performed:
1. Cut 6 pieces of mirrored glass according to the specifications for parts 14-02 to 14-07 (FIG. 18b).
2. Cut a piece in the shape of element 310b (FIG. 18b).
3. Core drill a 1.125″ hole into element 310b (FIG. 18b).
4. Assemble parts to form reflector structure 310 (FIG. 18). Mirrored surfaces of the mirrored glass face toward the inside the resulting structure 310. Any manner of techniques can be used to assemble these pieces but nothing should touch the inside surfaces of this assembly 310. Copper foil tape can be used on the outside surfaces to hold the assembly together and then a wooden box can be made to secure the whole assembly.
5. Build a copper cone 218 as specified in Par 17-02 of FIG. 10.
6. Mount this cone in a plywood frame supporting the lamp housing and connect to a wire to form ISF generator 304 as shown in Assembly 03 (FIG. 18a). The opening of the cone should remain open to the interior of the reflector without obstruction.

6. Ball Radiator Assembly

To construct a ball radiator assembly, reference is made to assembly Drawing 08, which is contained in FIG. 23.

In one embodiment of the present invention, the following exemplary steps are performed:
1. Using a copper ball 314 of about 0.625″ diameter solder 16 gauge wire to it of sufficient length so that it can be connected to the copper cone in the Lamp Assembly described above.
2. Cut a piece of wood 317 that is 6″ long by 1.5″ wide by 0.75″ thick.
3. Cut a 30 degree angle with the long side being 6″ and short side being 3″.
4. Cut a 1″ ID PVC tube 318 in the manner shown in the FIG. 23.
5. Mount the wooden piece to the PVC pipe, preferable with screws.
6. Use screws to mount a 9″ long ⅜″ diameter dowl 319 to the bottom of the wooden mount.
7. Attach a wire 316, preferably 16 gauge multi-strand wire, with copper ball 314, to the wooden dowel 319 with wire ties.

7. Main Assembly

To construct a main assembly, reference is made to FIGS. 2k, 3, 4, 5, 6, 7b, 2, 24 and 26, respectively. Further detailed description of respective parts is also provided below.

In one embodiment of the present invention, the following exemplary steps are performed:
1. Cut 6 1″ diameter copper piping 212 to 88″ in length, as illustrated in FIG. 2a.
2. Use copper foil tape to outline the pattern 208, as shown in FIG. 4. The hole diameter for a pattern of hexagonal through holes cut through an inner end member (204b of FIG. 4) is preferably about 1.125.″ Copper foil tape is preferably folded at its ends that form the star apices, such that the foil tape is folded into the 1.125″ diameter holes, in order to endure good contact with copper tubes 212 when the end of the tubes are placed into the holes. The junctions 204d of copper tape are preferably solder together, as illustrated in FIG. 4.
3. On an inner panel 204b for the bottom, solder 6 16 gauge wires to the tips 210 of the hexagon pattern.
4. Sandwich the inner and outer panels together, as illustrated in FIG. 2a. As illustrated in FIG. 3, make sure that an outer panel 204a having 6 wire feedthrough holes (204c, in the example shown in FIG. 3), is joined together with an inner panel (see panel 204b, FIG. 4) that has six wires soldered to the apices 210 copper foil hexagon pattern. The configuration described in steps 3-5 can be applied to both end members 204 and 206.
5. As illustrated in FIGS. 3 and 4, a 0.188″ diameter feed through hole is also provided in the center of the hexagonal patterns in panel 204a and 204b only, which provides for a wire connection to a foot panel.
6. Mount a bed 402 (see FIG. 5) to one of the panels 204, 206 using carriage bolts. Bed 402 preferably comprises a ¾ Luaun Surface finished plywood board, about 33″×86.5.″ The plywood board is supported by a series of three wood cross braces 402c about 1.5″×9.5″×19.75″ illustrated in FIG. 5. In addition, two 10″×86.5″ lengthwise braces 402d are used to support bed 202. The braces can be secured to bed 402 by 1.25″ deck screws spaced at 6″ intervals. In the embodiment of the present invention illustrated in FIG. 4, each end member has a height of about 53.64.″ The bottom side dimension is about 39.17″ and the top portion is beveled so that its width is about 13.188″ and the side edges have a height of about 46.64.″ The bed is mounted about 17.50″ above the bottom of board 204, which is above the line described by a pair of lower holes 210 that are 12.625″ above the lower surface of the end member.
7. Then the six copper tubes 212 are inserted into a first inside panel 204b or 206b. The outer four tubes are located 3″ from the front and back edges of the end member. The pair of upper holes is located 32.375″ above the lower surface of the end member, while the topmost hole is located 41.25″ above the lower surface. Accordingly, the center wire feedthrough is located about 2″ above the bed 202. As illustrated in FIG. 4, a slot 204e is provided on panel 204b so that one copper tube 212 disposed to the outside of panel 204b can be removed, thus promoting easy entry and egress to bed 402. Using the second of the inner-outer panels 206b, 204b align the 6 copper tubes 212 with the holes in the second panel, and mount the bed 202 using the appropriate carriage bolts.
8. Prepare a top assembly 424 comprising a top panel 424a about 12.75″×87.5″ as shown in FIG. 6. Support the top assembly using two 1.5″×3.5″×12.75″ cross braces 424b and two 0.75″×3.5″×86.5″ face strips 424cc, the latter preferably made from 0.75″ Luaun Surface Finished Plywood. Mount the top assembly 424 (see FIG. 6) to the support comprising bed 402 (also shown as 202 in FIG. 2k) and end panels 204, 206, and fasten assembly 424 in place using 0.375″ diameter carriage bolt holes provided in end members 204, 206, as illustrated in FIGS. 2k and 3.
9. As illustrated in FIGS. 24 and 26, place the hexagon assembly 216 on the top copper tube 212, making sure that the copper tube 212 fits well inside the two notches 216a (see FIG. 19) cut into the two ends of this assembly. In one embodiment, the hexagonal assembly support structure 216b comprises a set of 0.188″ thick polymer foam board pieces. The notches 216a are formed by cutting adjacent portions from abutting pieces of the hexagonal assembly to form 3.5″×1.125″ notches, as illustrated in FIG. 19. Use a suitable material to build a box 219 between the top of the hexagon assembly and the bottom of the top assembly 424, as illustrated in FIGS. 24 and 26. A Teflon™ sheet is then inserted between this box structure 219 and the Top assembly to reduce friction. The box structure 219 acts to keep assembly 216 parallel to bed 402. The hexagon assembly 216 can be enclosed with a suitable housing. Once complete, this hexagon assembly 216 should slide back and forth along the top copper tube 212 with ease, but the hexagon assembly 216 must remain parallel with the bed 402. Make sure that the center of the cones 218 in the hexagon assembly are about 27″ above the bed 402.
10. As illustrated in FIG. 23, a slot is cut in PVC tubing 318 to fit around the copper tubing 212. The ball radiator 312 is mounted by snapping the slotted PVC 318 over the top copper tube 212 between the hexagon assembly and the headboard.
11. Mount the IR camera 217 on the top assembly as shown in FIGS. 2k and 24. The IR camera is adjusted so that the head of an individual lying on foam mattress 203 is viewable on a monitor.
12. Mount the IR light source 215 with a suitable gooseneck mount as shown in FIGS. 2a and 24. The angle of light source 215 is adjusted to illuminate the head of an individual lying on mattress 203.

8. Setting up the Electronic Hardware

Below is an exemplary list of hardware used to generate electronic signals, and generate ISFs.

Parts list:
1. Philips CD player or any comparable CD player. See exemplary specification details above.
2. Yamaha or any comparable Sound Amplifier/Stereo receiver with greater than 30 W per channel of amplifier output. See exemplary specification details above.
3. Piezo Systems Linear Amplifier or any comparable High Voltage amplifier capable of taking a 10 V peak-to-peak signal in and outputting a minimum of 120V but not greater than 200V signal. The Piezo Systems Amplifier has a Bias offset which is necessary in this device. See exemplary specification details above.
4. ISF Generator: See construction details above.
5. Fiber Coupler: See construction details above.
6. Distribution Assembly: See construction details above.
7. Main Assembly with Hexagon projector and Ball Radiator Assembly are already installed.
8. SCR Lamp dimmer.
9. Lamp Module: See construction details above. Lamp ISF Generator illustrated in FIG. 18.

9. Connecting the Hardware

Below is an exemplary set of steps for connecting various hardware elements of a destressing apparatus, constructed in accordance with an embodiment of the present invention.
1. Referring again to FIG. 27a, connect the outputs of a CD player 272 to the Aux (or other comparable inputs) of the sound amplifier 276 using a standard phono jack cable 274.
2. Take a standard coax cable with BNC connectors that can be bought at any electronics store and cut one of the ends off. Take 16-gauge speaker wire and solder it to the core wire and another wire to the shielding. Connect the core wire to the positive speaker terminal and the shield wire to the negative speaker terminal (either right or left channel). Connect the other end with the BNC connector and attach it to the male BNC input connector on the front panel of the high voltage amplifier 278.
3. Take a length of coax wire with BNC connectors that is long enough to go from the high voltage amplifier 278 to the ISF generator 282, which is placed close to the main assembly. Cut one of the BNC connectors off and attach banana plugs to the positive and negative wires and insert the banana plugs into the positive and negative output terminals on the front panel of the high voltage amplifier 278. Take the other BNC connector and connect it to the female BNC connector on the ISF generator 282.
4. Connect the switch 280 on the ISF generator terminal between the negative end of the bulk head BNC connector and the outside bronze/Teflon™ capacitor 324 (FIG. 8b). Make sure that the positive input of the bulk head BNC connector is connected to the inner bronze/Teflon™ capacitor. Use appropriate solder to make both connections.
5. Referring again to FIG. 8b, take a 16 gauge multi-strand wire and connect it to the tip of the copper cone 328 on the ISF generator. This can be done by either soldering the wire to the tip of the cone 328 or by solder a ring connector to the wire and using the appropriate screw to tighten the ring connector to the tip of the cone. The other end of this wire is connected to the input of the fiber coupler assembly 286 (FIG. 27a). It is possible to use a male and female connector in the wire between the ISF generator and the fiber coupler, but any connector used must not have lead based solder. Copper connectors with no solder are preferable, but nickel plated connectors will also work. It can be seen from FIG. 16 that the input and output wire of the fiber coupler 286 are attached to the tip of the copper cones in the manner shown in FIG. 16.
6. Referring again to FIG. 27a, use the same 16 gauge wire to make a connection from the output of the fiber coupler 286 to the input of the distribution assembly 340. The input wire of the distribution assembly is connected to the tip of the cone in this assembly. Again, wire attachment is done in the same manner as shown in the drawing for FIG. 16. The 6 output wires are soldered to the 6 tips of the base of the distribution assembly. Each of these 6 wires is connected to one of the cones on the hexagon projector 290, as depicted in FIG. 27a.
7. The connections for the lamp assembly 304 are made as follows. A 110V power supply cord is connected to the lamp socket base. This cord is then plugged into the output of the SCR dimmer control.
8. Referring again to FIG. 2k, the video output plug from the IR camera 217 is connected to video input of any TV purchase for this device. The IR camera as specified comes with a separate transformer for DC power.

10. Setting Up and Optimizing the Electronics

Below is an exemplary set of steps for setting up and optimizing the electronic components of a destressing system, according to one embodiment of the present invention.
1. Insert a CD into the CD player 272.
2. Use an oscilloscope to measure the speaker output of the sound amplifier 276. Adjust the volume control until the speaker output has a median value of 5 V peak-to-peak and should not exceed 10 V peak-to-peak during any portion of the music.
3. Before turning on the high voltage (HV) amplifier 278, turn the gain to minimum. Hook up the ISF generator 282 to the HV amplifier 278. Make sure the switch 280 on the ISF generator is off. Use an oscilloscope to measure the output of the HV amplifier. Set the Bias Polarity to positive. Adjust the Bias offset so that there is a positive 150 V bias. Now turn up the gain and make sure that at no point during does the signal go below zero volts DC. If this does happen, the more Bias offset needs to be applied. Once the bias set, turn the gain back down and this part of the electronics is ready.
4. Plug the IR camera supply into a power strip. Once this is turned on, turn the IR light 215 on and the system is ready to be optimize to the person in the resting in the device.
5. Plug the SCR into the same power strip as the IR camera. As power switch on the front panel of the SCR is used to turn it on.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents. For example, although embodiments of the invention disclosed above focus on apparatus in which an individual is accommodated in a reclined position, apparatus in which the end members are arranged so that the longitudinal members are vertical and the individual is upright during destressing are within the scope of the invention. Additionally, component or apparatus dimensions discussed in the text or indicated in the Figures are merely exemplary and not meant to limit the scope of the invention.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A system for reducing stress, comprising:
a bed configured to accommodate an individual thereon;
an electromagnetic source for generating an electrical or electromagnetic signal; an axially symmetric generating body configured to receive an electrical or electromagnetic signal or both from the electromagnetic source; and
a projector coupled to the generating body and configured to establish one or more fields that substantially surround at least a portion of the individual when the individual is on the bed, wherein the system is configured to reduce stress when the generating body is receiving an electrical or electromagnetic signal.

2. The system of claim 1, wherein the electromagnetic source comprises one of a system for generating a signal derived from music and a signal generator.

3. The system of claim 2, wherein the system for generating a signal derived from music comprises:
a device for playing a music recording;
a receiver configured to receive a signal from the device for playing a music recording; and
one or more amplifiers configured to receive an output from the receiver and to output an electric signal to the generating body.

4. The system of claim 1, wherein the generating body comprises:
one or more outer capacitor layers arranged in cylindrical form;
a magnetic body disposed in a ring shape within the one or more outer capacitor layers;
one or more inner capacitor layers concentrically disposed within the ring shape of magnetic material;
a support structure configured to support the one or more outer capacitor layers, one or more outer inner capacitor layers and magnetic body; and
a conical receiving element affixed to the metallic housing, wherein the generating body is configured to produce a fluctuating magnetic field when a varying voltage signal is received from the one or more amplifiers.

5. The system of claim 4, wherein the magnetic body has a north pole oriented along an axis of the ring and capacitors, wherein the north pole faces toward the conical receiving element.

6. The system of claim 4, wherein the conical receiving element has a base whose dimension is about a factor of 1.618 times that of its height.

7. The system of claim 1, further comprising an attenuator configured to block electromagnetic and/or electric signals received or generated by the generating body from impinging on the individual, wherein the attenuator comprises:
a first coupler electrically connected to the generating body and configured to block transmission of any electrical signals received from or induced during transmission from the generating body; and
a second coupler configured to block transmission of electromagnetic radiation from propagating from the generating body to the projector,
wherein the first and second coupler each comprise a pair of opposed cones whose bases are aligned,
wherein each pair comprises a metallic cone and a cone made of insulating material, and wherein each cone made of insulating material supports on its outer surface a glass fiber wound thereon, the glass fiber forming a continuous path between the cones made of insulating material.

8. The system of claim 1, wherein the generating body comprises a lamp module and the electromagnetic source comprises an SCR controller configured to generate a voltage that does not vary according to an AC line frequency,
wherein the lamp module comprises:
an incandescent light source coupled to the SCR;
an opaque housing that surrounds the incandescent light source; and
a metallic cone configured to receive light from the incandescent light source and
to substantially block light from exiting the housing.

9. The system of claim 8, further comprising a hexagonal reflector configured to generate a static field.

10. The system of claim 8, wherein the projector comprises:
a ball radiator comprising a conductive material; and
a conductive wire strand connected to the ball radiator and lamp module.

11. The system of claim 1, wherein the projector comprises:
a hexagonal radiator cone assembly having six metallic cones approximately equally spaced along an outer surface of a hexagonal structure, each cone having an apex disposed outwardly from the hexagonal structure, wherein a cone axis of each cone approximately intersects at a common point with all other cone axes of all other cones of the cone assembly, the common point located within a region between the projector and bed; and
a hexagonal distributor having a central conical structure coupled through its apex to the generating body, the hexagonal distributor further comprising a star plate having six apices that are each coupled to a respective one of the six metallic cones.

12. The system of claim 1, further comprising a monitoring system that comprises:
an IR source located within the chamber and configured to provide radiation substantially in a non-visible frequency range; and
an IR detector configured to receive IR radiation form the individual and to provide a visible image of the individual to a display.

13. The system of claim 1, further comprising:
a pair of end members that are each connected to the bed on opposite ends of the bed;
a set of six longitudinal members each of whose ends contact a respective end member, the metallic tubes being mutually parallel and forming a hexagonal array as viewed along their axis;
a foot assembly comprising a metallic surface plate region configured to support feet; and
a hexagonal concentrator having a base portion comprising a set of six apices, each apex coupled to a respective metallic tube, the concentrator further comprising a cone joined at its base to the base portion, wherein the cone apex is coupled to the metallic surface plate.

14. The system of claim 23, wherein one or more end members comprise:
a pair of electrically insulating boards joined together; and
a Star of David pattern comprising a conductive material and disposed between the boards, wherein each apex of the pattern is coupled to an end of a respective metallic tube.

15. A system for reducing stress by providing a beneficial ISF environment, comprising:
a support structure configured to receive an individual;
an electrical signal generator configured to generate at least one of an electric and electromagnetic signal;
an ISF generator configured to generate one or more ISFs based at least in part upon a an electrical or electromagnetic signal received from the electrical signal generator;
a conductor configured to conduct the one or more ISFs from the ISF generator; and
an ISF projector system configured to distribute the one or more ISFs at least in a portion of a region surrounding the individual when the individual is located on the support structure.

16. The system of claim 15, wherein the one or more ISFs comprises at least one statically generated ISF and at least one dynamically generated ISF.

17. The system of claim 16, further comprising:
a coupler configured to block transmission of at least one of an electromagnetic and electric signal from being transmitted from the ISF generator to the ISF projector system;
a set of six elongated metallic tubes connected to the support structure, arranged horizontally, being mutually parallel and forming a hexagonal array as viewed along their axes;
a foot assembly comprising a metallic surface plate region configured to support feet;
a hexagonal concentrator having a base portion comprising a set of six apices, each apex coupled to a respective metallic tube, the concentrator further comprising a cone joined at its base to the base portion, wherein the cone apex is coupled to the metallic surface plate;
wherein the ISF projector system comprises:
a hexagonal cone assembly disposed over the support structure and having six metallic cones approximately equally spaced along an outer surface of a hexagonal structure, each cone having an apex disposed outwardly form the hexagonal structure; and
a ball irradiator structure disposed over the bed and comprising a metallic sphere, wherein the hexagonal irradiator and ball radiator are configured to each project a dynamically derived ISF into a region surrounding the individual when the ISF generator receives an electric or electromagnetic signal.

18. The system of claim 1, further comprising a pair of end members that are each connected to the bed on opposite ends of the bed, wherein one or more end members comprise:
a plurality of electrically insulating boards joined together; and
a Star of David pattern comprising a conductive material and disposed between two of the plurality of electrically insulating boards, wherein each apex of the pattern is coupled to an end of a respective metallic tube.

19. A system for alleviating or reducing stress in an individual comprising an informational spin field environment substantially surrounding an individual which is at least partially derived from one or more dynamically produced informational spin fields wherein electromagnetic components associated with producing one or more of such dynamically produced informational spin fields have first been substantially separated therefrom, such one or more dynamically produced informational spin fields being then conducted to the environment substantially surrounding the individual.

20. A method for alleviating or reducing stress in an individual, comprising providing an informational spin field environment substantially surrounding such individual which is at least partially derived from one or more dynamically produced informational spin fields wherein electromagnetic components associated with producing one or more of such dynamically produced informational spin fields have first been substantially separated therefrom, such one or more dynamically produced informational spin fields being then conducted to the environment substantially surrounding the individual.
